(19) United States
(12) Patent Application Publication
(10) Pub. No.: US 2017/0188927 A1
(43) Pub. Date: Jul. 6, 2017

(54) EMOTION RECOGNITION DEVICE, EMOTION RECOGNITION METHOD, AND STORAGE MEDIUM FOR STORING EMOTION RECOGNITION PROGRAM

(71) Applicant: NEC Corporation, Tokyo (JP)

(72) Inventors: Yoshiki NAKASHIMA, Toyko (JP); Mitsuru SENDODA, Tokyo (JP)

(73) Assignee: NEC Corporation, Tokyo (JP)

(21) Appl. No.: 15/313,154

(22) PCT Filed: May 20, 2015
(86) PCT No.: PCT/JP2015/002541

§ 371 Date: Nov. 22, 2016

(30) Foreign Application Priority Data
May 27, 2014 (JP) 2014-109015

(51) Int. Cl. A61B 5/16; A61B 5/024; G06N 99/00; A61B 5/08; A61B 5/00; A61B 5/01; A61B 5/0295

(52) U.S. Cl. CPC A61B 5/165; A61B 5/01; A61B 5/024; A61B 5/0295; A61B 5/0816; A61B 5/7267; G06N 99/005

(57) ABSTRACT

An emotion recognition device includes: a memory that stores a set of instructions; and at least one processor configured to execute the set of instructions to: classifies, based on a second emotion, a biological information pattern variation amount indicating a difference between first biological information and second biological information, the first biological information being measured by sensor from a test subject in a state in which a stimulus for inducing a first emotion is applied, the second biological information being measured in a state in which a stimulus for inducing the second emotion; and learn a relation between the biological information pattern variation amount and each of a plurality of emotions as the second emotion in a case where the biological information pattern variation amount is obtained, based on a result of classification of the biological information pattern variation amount.

### TECHNICAL FIELD

The present invention relates to a technology of estimating an emotion, and particularly to a technology of estimating an emotion using biological information.

### BACKGROUND ART

Methods using, as clues, biological information reflecting the activities of autonomic nervous system, such as a body temperature, a heart rate, the skin conductance, a respiratory frequency, and a blood flow volume, are widely known as methods of estimating the emotions of test subjects. Generally, when estimating the emotions of the test subjects with biological information, stimuli inducing specific emotions are applied to test subjects by methods such as moving images, music, and pictures. Then, the values of the biological information of the test subjects in that state are recorded. Emotion recognition devices learn the combinations (i.e., biological information patterns) of the values of the biological information due to the specific emotions based on the recorded values of the biological information by supervised machine learning methods. The emotion recognition devices estimate the emotions based on the results of the learning.

However, there are variations in the absolute values of biological information among individuals. For example, body temperatures and heart rates at rest vary among individuals. Therefore, the emotion recognition device described above is not necessarily capable of performing estimation with high accuracy when a test subject whose biological information is recorded and used for learning using a supervised learning method and a test subject whose emotion is estimated are different from each other. Technologies of generalizing such emotion recognition devices using supervised learning methods (i.e., technologies of making it possible to adapt such emotion recognition devices not only to specific users but also to, for example, an unspecified large number of users) are disclosed in, for example, NPL 1 and NPL 2. In the technologies disclosed in NPL 1 and NPL 2, the absolute value of biological information due to a specific emotion is not measured but a change (relative value) between biological information patterns at rest and at the time of application of a stimulus for inducing the specific emotion is measured.

An example of a technology of identifying an emotion based not on a change from the absolute value of biological information at rest but on changes in feature quantities of biological information is disclosed in PTL 1. In an emotion detection apparatus described in PTL 1, information in which patterns of changes in feature quantities of biological information are associated with emotions is stored in advance in an emotion database. In an example described in PTL 1, the changes in the feature quantities of the biological information are changes in the intensity, tempo, and intonation in words of voice emitted by a test subject. The emotion detection apparatus estimates that an emotion associated with a pattern of a change in a detected feature quantity of biological information in the information stored in the emotion database is the emotion of a test subject with the detected biological information.

### CITATION LIST

### Patent Literature

[PTL 1] Japanese Unexamined Patent Application Publication No. 2002-091482

### Non Patent Literature

[NPL 1] Rosalind W. Picard, Elias Vyzas and Jennifer Healey, “Toward Machine Emotional Intelligence: Analysis of Affective Physiological State,” IEEE Transactions on Pattern Analysis and Machine Intelligence, Vol. 23, No. 10, pp. 1175-1192, 2001

[NPL 2] Jonghwa Kim and Elisabeth Andre, “Emotion Recognition Based on Physiological Changes in Music Listening,” IEEE TRANSACTIONS ON PATTERN ANALYSIS AND MACHINE INTELLIGENCE, VOL. 30, NO. 12, PP. 2067-2083, DECEMBER 2008

### SUMMARY OF INVENTION

### Technical Problem

Biological information used as a clue for estimating an emotion is reflected with the activities of autonomic nervous system. Therefore, biological information fluctuates depending on the activities of autonomic nervous system (for example, digestion and absorption, adjustment of body temperature, and the like) even when a test subject is at rest. Even when a test subject is directed to be at rest, the test subject may have a specific emotion. Therefore, methods for estimating an emotion based on, as a clue, a variation from a biological information pattern at rest, which is considered to be a baseline, the methods being known as common methods, are limited. In other words, the biological information of a test subject at rest is not always the same. Further, the emotion of a test subject at rest is not always the same. Accordingly, when an emotion recognition reference is biological information obtained at rest, it is difficult to eliminate fluctuations in the reference. Accordingly, it is not always possible to estimate an emotion with high accuracy by the methods of estimating an emotion based on biological information at rest as a reference, as described in NPLs 1 and 2.

In the emotion detection apparatus described in PTL 1, the patterns of the changes in the feature quantities of the biological information are associated with changes from specific emotions to other specific emotions. However, a method of covering all the possible combinations of the emotions before changes (emotions as references) and the emotions after the changes (emotions targeted for identification) is not described. When a certain emotion targeted for identification is intended to be reliably identified by the method of PTL 1, emotions as references need to be all emotions other than the emotion targeted for identification. Therefore, the emotion detection apparatus described in PTL 1 is not always capable of identifying all emotions with high accuracy.

One of the objects of the present invention is to provide an emotion recognition device and the like by which a decrease in the accuracy of identification of an emotion due to fluctuations in an emotion recognition reference can be suppressed.

### Solution to Problem

An emotion recognition device according to one aspect of the present invention includes: classification means for classifying, based on a second emotion, a biological information pattern variation amount indicating a difference between biological information measured by sensing means from a test subject in a state in which a stimulus for inducing a first emotion is applied, the first emotion being one of two emotions obtained from a plurality of combinations of two different emotions from among a plurality of emotions, and the biological information measured in a state in which a stimulus for inducing the second emotion which is the other of the two emotions is applied after the biological information is measured; and learning means for learning a relation between the biological information pattern variation amount and each of the plurality of emotions as the second emotion in a case where the biological information pattern variation amount is obtained, based on the result of classification of the biological information pattern variation amount.

An emotion recognition method according to one aspect of the present invention includes: classifying, based on a second emotion, a biological information pattern variation amount indicating a difference between biological information measured by sensing means from a test subject in a state in which a stimulus for inducing a first emotion is applied, the first emotion being one of two emotions obtained from a plurality of combinations of two different emotions from among a plurality of emotions, and the biological information measured in a state in which a stimulus for inducing the second emotion which is the other of the two emotions is applied after the biological information is measured; and learning a relation between the biological information pattern variation amount and each of the plurality of emotions as the second emotion in a case where the biological information pattern variation amount is obtained, based on the result of classification of the biological information pattern variation amount.

A recording medium according to one aspect of the present invention stores an emotion recognition program that operates a computer as: classification means for classifying, based on a second emotion, a biological information pattern variation amount indicating a difference between biological information measured by sensing means from a test subject in a state in which a stimulus for inducing a first emotion is applied, the first emotion being one of two emotions obtained from a plurality of combinations of two different emotions from among a plurality of emotions, and the biological information measured in a state in which a stimulus for inducing the second emotion which is the other of the two emotions is applied after the biological information is measured; and learning means for learning a relation between the biological information pattern variation amount and each of the plurality of emotions as the second emotion in a case where the biological information pattern variation amount is obtained, based on the result of classification of the biological information pattern variation amount. The present invention can also be accomplished by the emotion recognition program stored in the recording medium described above.

### Advantageous Effects of Invention

The present invention has an advantage in that a decrease in the accuracy of identification of an emotion due to fluctuations in an emotion recognition reference can be suppressed.

### BRIEF DESCRIPTION OF DRAWINGS

FIG. 1 is a block diagram representing an example of a configuration of an emotion recognition system 201 according to a comparative example.

FIG. 2 is a block diagram representing an example of a configuration of an emotion recognition device 101 of the comparative example.

FIG. 3 is a block diagram representing an example of a configuration of the emotion recognition system 201 of the comparative example in a learning phase.

FIG. 4 is a block diagram representing an example of a configuration of the emotion recognition device 101 of the comparative example in a learning phase.

FIG. 5 is a second block diagram representing an example of a configuration of the emotion recognition device 101 of the comparative example in a learning phase.

FIG. 6 is a view representing an example of classified emotions.

FIG. 7 is a block diagram representing an example of a configuration of the emotion recognition system 201 of the comparative example in an estimation phase.

FIG. 8 is a block diagram representing an example of the configuration of the emotion recognition device 101 of the comparative example in an estimation phase.

FIG. 9 is a flowchart representing an example of an operation of the emotion recognition system 201 of the comparative example in a learning phase.

FIG. 10 is a flowchart representing an example of an operation of processing of extracting a biological information pattern variation amount by the emotion recognition system 201 of the comparative example.

FIG. 11 is a flowchart representing an example of a first operation of the emotion recognition device 101 of the comparative example in a learning phase.

FIG. 12 is a flowchart representing an example of a second operation of the emotion recognition device 101 of the comparative example in a learning phase.

FIG. 13 is a flowchart representing an example of an operation of the emotion recognition system 201 of the comparative example in a determination phase.

FIG. 14 is a view representing an example of an operation of the emotion recognition device 101 of the comparative example in a determination phase.

FIG. 15 is a block diagram representing an example of a configuration of an emotion recognition system 2 of a first exemplary embodiment of the present invention.

FIG. 16 is a block diagram representing an example of a configuration of the emotion recognition system 2 of the first exemplary embodiment of the present invention in a learning phase.

FIG. 17 is a block diagram representing an example of a configuration of the emotion recognition system 2 of the first exemplary embodiment of the present invention in an estimation phase.

FIG. 18 is a block diagram representing an example of a configuration of an emotion recognition device 1 of the first exemplary embodiment of the present invention.

FIG. 19 is a first block diagram representing an example of a configuration of the emotion recognition device 1 of the first exemplary embodiment of the present invention in a learning phase.

FIG. 20 is a second block diagram representing an example of a configuration of the emotion recognition device 1 of the first exemplary embodiment of the present invention in a learning phase.

FIG. 21 is a view schematically representing processing of a first distribution formation unit 11, a synthesis unit 12, and a second distribution formation unit 13 of the first exemplary embodiment of the present invention.

FIG. 22 is a block diagram representing an example of a configuration of the emotion recognition device 1 of the first exemplary embodiment of the present invention in an estimation phase.

FIG. 23 is a flowchart representing an example of an operation of the emotion recognition system 2 of the first exemplary embodiment of the present invention in a learning phase.

FIG. 24 is a flowchart representing an example of the operation of processing for extracting a relative value of a biological information pattern by the emotion recognition system 2 of the first exemplary embodiment of the present invention.

FIG. 25 is a first flowchart representing an example of an operation of the emotion recognition device 1 of the first exemplary embodiment of the present invention in a learning phase.

FIG. 26 is a first flowchart representing an example of an operation of the emotion recognition device 1 of the first exemplary embodiment of the present invention in a learning phase.

FIG. 27 is a flowchart representing an example of an operation of the emotion recognition system 2 of the first exemplary embodiment of the present invention in an estimation phase.

FIG. 28 is a flowchart representing an example of an operation of the emotion recognition device 1 of the first exemplary embodiment of the present invention in an estimation phase.

FIG. 29 is a view schematically representing patterns in a one-dimensional subspace in a feature space in the comparative example.

FIG. 30 is a view schematically representing patterns in a one-dimensional subspace in a feature space of the first exemplary embodiment of the present invention.

FIG. 31 is a view schematically representing a distribution of biological information pattern variation amounts obtained in each emotion in the first exemplary embodiment of the present invention.

FIG. 32 is a view representing an example of classification of emotions.

FIG. 33 is a block diagram representing a configuration of an emotion recognition device 1A of a second exemplary embodiment of the present invention.

FIG. 34 is a block diagram representing an example of a configuration of a computer 1000 by which an emotion recognition device 1, an emotion recognition device 1A, and an emotion recognition system 2 can be achieved.

### DESCRIPTION OF EMBODIMENTS

Exemplary embodiments of the present invention are described in detail below. Although the exemplary embodiments described below are technologically preferably limited for carrying out the present invention, the scope of the invention is not limited to the following.

First, a comparative example is described so that differences between the comparative example and the exemplary embodiments of the present invention based on variations from biological information at rest become clear. Then, the exemplary embodiments of the present invention are described.

### Comparative Example

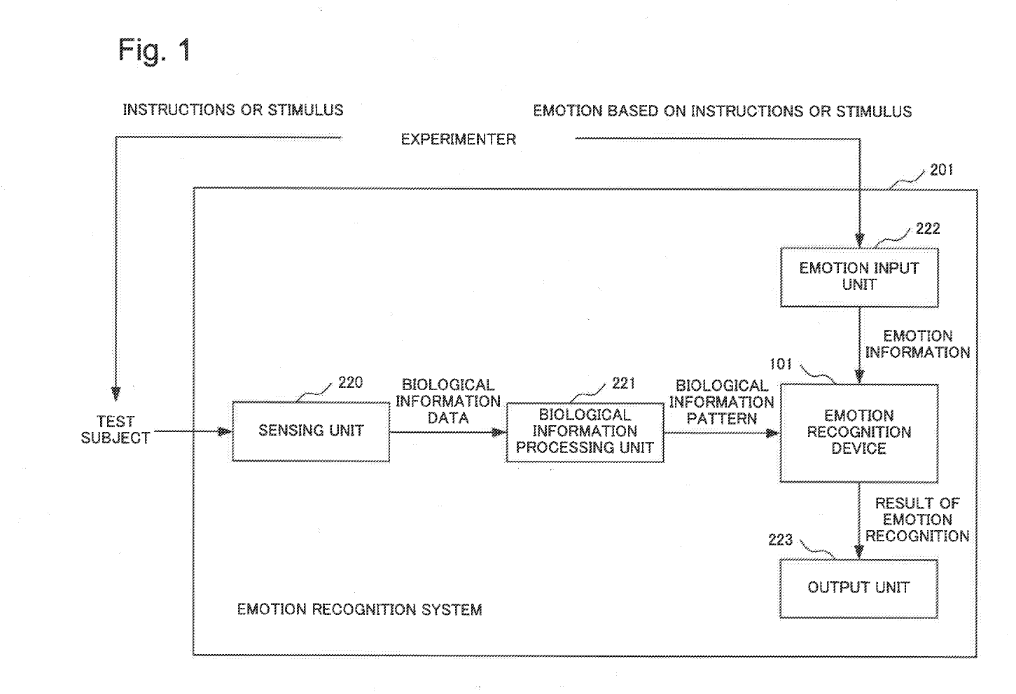

FIG. 1 is a block diagram representing an example of a configuration of an emotion recognition system 201 according to the comparative example.

According to FIG. 1, the emotion recognition system 201 includes a sensing unit 220, a biological information processing unit 221, an emotion input unit 222, an emotion recognition device 101, and an output unit 223. In the example illustrated in FIG. 1, the emotion recognition system 201 is drawn as one device including the emotion recognition device 101. However, the emotion recognition system 201 may be implemented using a plurality of devices. For example, the emotion recognition system 201 may be implemented using: a measuring device (not illustrated) including the sensing unit 220, the biological information processing unit 221, the emotion input unit 222 and the output unit 223, and the emotion recognition device 101. In this case, the measuring device and the emotion recognition device 101 may be communicably connected to each other.

The sensing unit 220 measures the plural kinds of the biological information of a test subject. Examples of such items of biological information include a body temperature, a pulse rate per unit time, a respiratory rate per unit time, skin conductance, and a blood pressure. The biometric information may be other information. The sensing unit 220 is implemented with either or the combination of both of a contact-type sensing device for measuring biological information in the state of being in contact with the skin of a test subject and a non-contact-type sensing device for measuring biological information in the state of not being in contact with the skin of the test subject. Examples of the contact-type sensing device include a type of body temperature sensor affixed to a skin surface, a skin conductance measurement sensor, a pulse sensor, and a type of respiration sensor wrapped around the abdomen or the chest. Examples of the non-contact-type sensing device include a body temperature sensor with an infrared camera, and a pulse sensor with an optical camera. The contact-type sensing device has an advantage in that detailed data can be collected with high accuracy. The non-contact-type sensing device has an advantage in that the device puts a small burden upon a test subject because it is not necessary to affix the device to a skin surface or to wrap the device around the trunk. In the following description, data representing biological information, obtained by measurement of biological information, is also referred to as “biological information data”.

The biological information processing unit 221 extracts a feature quantity representing biological information from the data of the biological information measured by the sensing unit 220. First, the biological information processing unit 221 may remove noise. The biological information processing unit 221 may extract a waveform in a specific wavelength band, for example, from the data of the biological information fluctuating depending on time. The biological information processing unit 221 may include a band-pass filter that removes noise and that extracts data having a specific wavelength, for example, from the periodically varying data values of biological information. The biological information processing unit 221 may include an arithmetic processing unit that extracts statistics such as, a mean value and a standard deviation of biological information within a specific time width. Specifically, the biological information processing unit 221 extracts feature quantities such as, a specific wavelength component, and statistics such as a mean value and a standard deviation from raw data of biological information such as, a body temperature, a heart rate, the skin conductance, a respiratory frequency, and a blood flow volume. The extracted feature quantities are used in machine learning by the emotion recognition device 101. In the following description, the combinations of all the feature quantities used by the emotion recognition device 101 are referred to as “biological information pattern”. A space spanned by all the feature quantities included in the biological information pattern is referred to as “feature space”. The biological information pattern occupies one point of the feature space.

First, for example, the biological information processing unit 221 extracts a biological information pattern from biological information data measured while the state of a test subject is a resting state. In the following description, the biological information pattern extracted from the biological information data measured while the state of the test subject is the resting state is also referred to as “resting pattern”. A time during which a test subject is in a resting state is also referred to as “at rest”. The biological information processing unit 221 may specify, for example, biological information data measured while the state of a test subject is a resting state, based on instructions from an experimenter. The biological information processing unit 221 may consider, for example, biological information measured during a predetermined time period from the start of the measurement to be the biological information data measured while the state of the test subject is the resting state.

The biological information processing unit 221 further extracts a biological information pattern from biological information data measured in a state in which a stimulus for inducing an emotion is applied to a test subject. In the following description, the biological information pattern extracted from the biological information data measured in the state in which the stimulus for inducing an emotion is applied to the test subject is also referred to as “stimulation pattern”. The biological information processing unit 221 may specify, based on, for example, instructions from an experimenter, the biological information data measured in the state in which the stimulus for inducing an emotion is applied to the test subject. The biological information processing unit 221 may detect a change in a biological information pattern. The biological information processing unit 221 may consider a biological information pattern measured before the detected change to be biological information data measured while the state of the test subject is a resting state. Further, the biological information processing unit 221 may consider a biological information pattern measured after the detected variation to be the biological information data measured in the state in which the stimulus for inducing an emotion is applied to the test subject.

The biological information processing unit 221 may send a resting pattern and a stimulation pattern to the emotion recognition device 101. In this case, for example, a receiving unit 116 in the emotion recognition device 101 may calculate a relative value of a biological information pattern, described later, representing a change from the resting pattern to the stimulation pattern. The biological information processing unit 221 may calculate the relative value of the biological information pattern. The biological information processing unit 221 may send the relative value of the biological information pattern to the emotion recognition device 101. In the following description, the biological information processing unit 221 calculates the relative value of a biological information pattern, and sends the calculated relative value of the biological information pattern to the emotion recognition device 101.

The emotion recognition device 101 is a device that carries out supervised machine learning as described below. Combinations of derived biological information patterns and emotions, induced by stimuli applied to a test subject when biological information data are measured, from which the biological information patterns are derived, are, for example, repeatedly input to the emotion recognition device 101. A plurality of combinations of biological information patterns and emotions, repeatedly acquired in advance, may be input in a lump to the emotion recognition device 101. The emotion recognition device 101 learns relations between the biological information patterns and the emotions based on the input combinations of the biological information patterns and the emotions, and stores the results of the learning (learning phase). When a biological information pattern is further input, the emotion recognition device 101 estimates the emotion of a test subject in the case of obtaining the relative value of the input biological information pattern, based on the results of the learning (estimation phase).

The emotion input unit 222 is a device by which, for example, an experimenter inputs emotion information into the emotion recognition device 101 in a learning phase. The emotion input unit 222 is a common input device such as, a keyboard or a mouse. The output unit 223 is a device by which the emotion recognition device 101 outputs an emotion recognition result in an estimation phase. The output unit 223 may be a common output device such as a display. The output unit 223 may be a machine such as a consumer electrical appliance or an automobile, operating depending on an emotion recognition result. The experimenter inputs a data value specifying an emotion to the emotion recognition system 201, for example, by manipulating the emotion input unit 222. The emotion input unit 222 sends, to the emotion recognition device 101, emotion information representing the emotion specified by the data value input by the experimenter. The emotion information is, for example, an emotion identifier specifying an emotion. In the description of the present comparative example and each exemplary embodiment of the present invention, inputting a data value for specifying an emotion is also referred to as “inputting emotion”. Sending emotion information is also referred to as “sending emotion”.

The states of the emotions of a test subject variously vary depending on an applied stimulus. The states of the emotions can be classified into a set of the states of the emotions depending on the characteristics of the states. In the present comparative example and each exemplary embodiment of the present invention, an emotion represents, for example, a set into which a state of emotion of a test subject is classified. “Stimulus for inducing emotion” applied to a test subject may be, for example, a stimulus, for example, experimentally known in advance to be very likely to allow a state of emotion of a test subject to which the stimulus is applied to be a state included in a set represented by the emotion. The set into which a state of emotion of a test subject is classified is described in detail later. An experimenter may select an appropriate emotion from a plurality of emotions which are determined in advance. The experimenter may input the selected emotion.

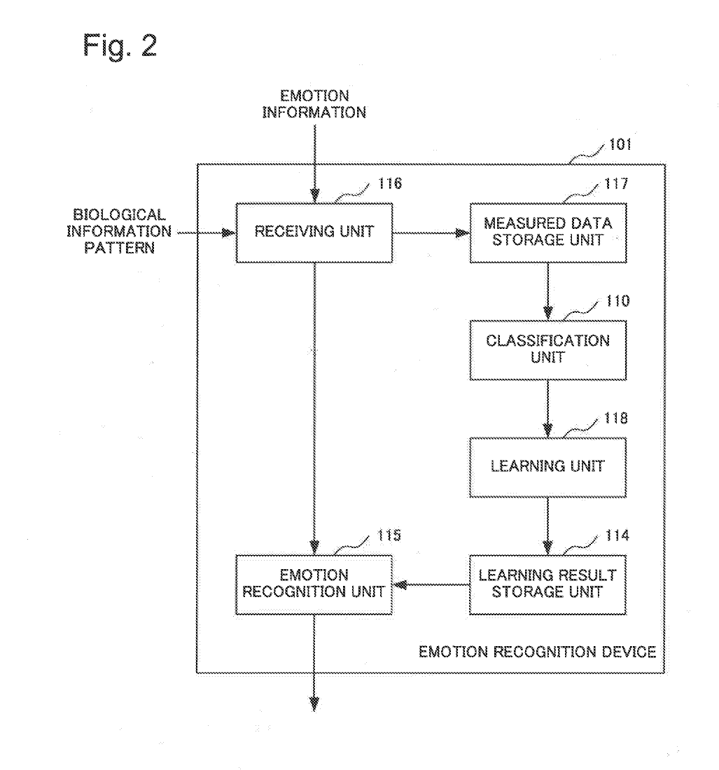

FIG. 2 is a block diagram representing an example of a configuration of the emotion recognition device 101 of the present comparative example. According to FIG. 2, the emotion recognition device 101 includes the receiving unit 116, a measured data storage unit 117, a classification unit 110, a learning unit 118, a learning result storage unit 114, and an emotion recognition unit 115.

The emotion recognition system 201 and the emotion recognition device 101 in a learning phase are described next in detail with reference to the drawings. In the learning phase, a test subject whose biological information is measured is not limited to a particular test subject. An experimenter manipulating the emotion recognition system 201 may measure, for example, the biological information of a number of test subjects who are not limited to a particular test subject.

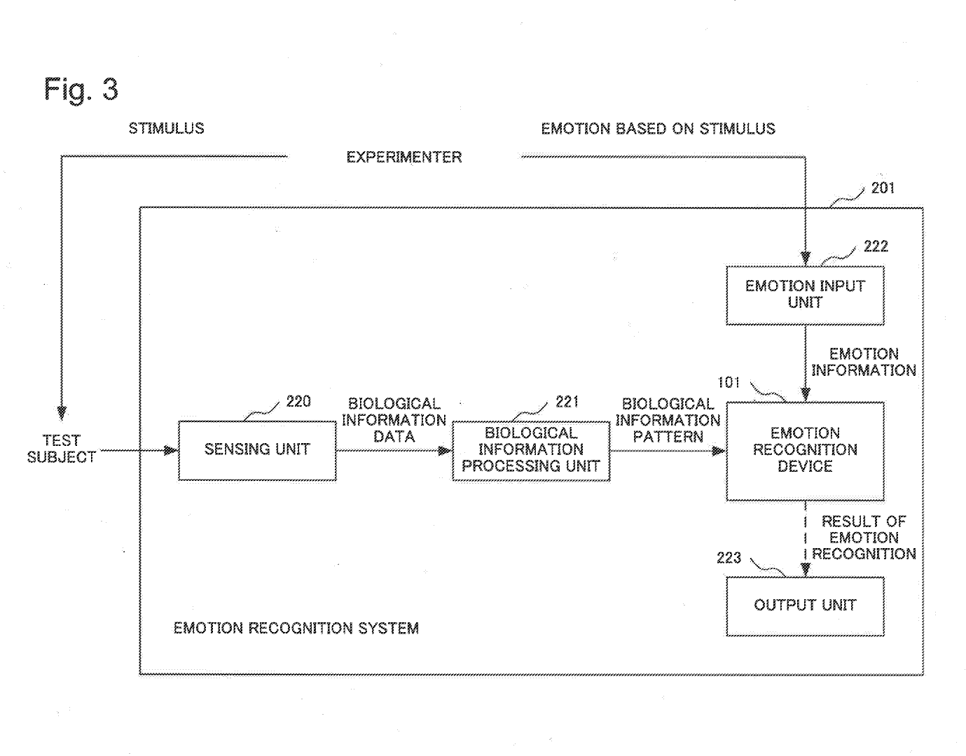

FIG. 3 is a block diagram representing an example of a configuration of the emotion recognition system 201 a learning phase. In the learning phase, an experimenter starts measurement of the biological information of a test subject by the sensing unit 220 of the emotion recognition system 201 in a state in which any stimulus is not applied to the test subject. The experimenter may be a system constructor constructing the emotion recognition system 201. The experimenter himself/herself may be the test subject. The experimenter may provide instructions to be at rest to the test subject, and may then start the measurement of the biological information of the test subject. After the start of the measurement, the experimenter applies a stimulus for inducing a specific emotion to the test subject. The stimulus is, for example, a voice or an image. The experimenter further inputs an emotion induced by the stimulus applied to the test subject, to the emotion recognition device 101 by the emotion input unit 222. The emotion induced by the stimulus applied to the test subject is, for example, is an emotion experimentally confirmed to be induced in the test subject or to be more likely to be induced in the test subject by the stimulus applied to the test subject. The emotion input unit 222 sends, to the emotion recognition device 101, emotion information representing the emotion input by the experimenter. By the operation described above, the sensing unit 220 measures the biological information during a time period from when the state of the test subject is a state in which the test subject is at rest until when the state of the test subject becomes a state in which the specific emotion is induced in the test subject by applying the stimulus to the test subject. In other words, the sensing unit 220 can acquire the variation amount (i.e., relative value) of the measured values of the biological information (i.e., biological information data) in a case in which the state of the test subject is changed from the resting state to the state of having the specific emotion. The biological information processing unit 221 derives a biological information pattern variation amount (i.e., relative value of a biological information pattern) by processing the biological information data acquired by the sensing unit 220. The biological information processing unit 221 inputs the relative value of the biological information pattern into the emotion recognition device 101.

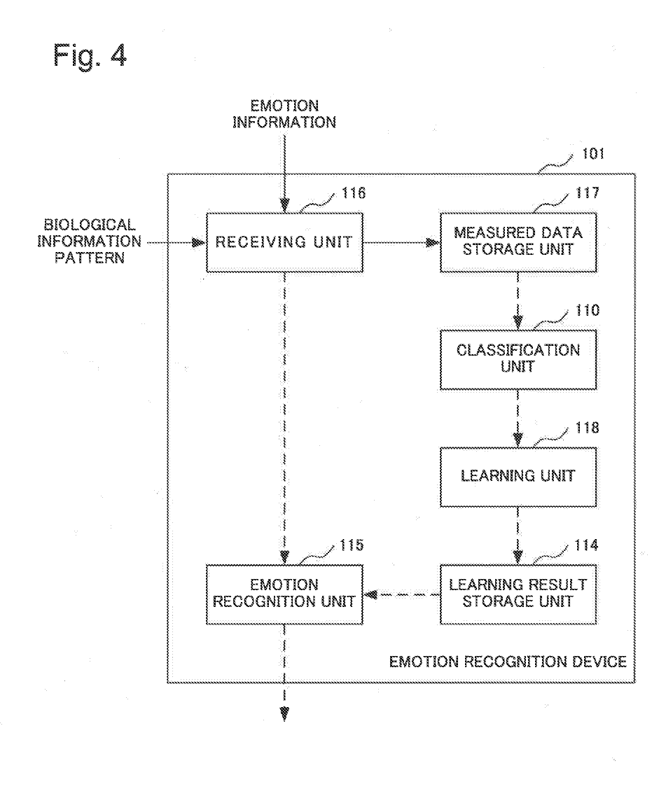

FIG. 4 is a block diagram representing an example of a configuration of the emotion recognition device 101 in a learning phase. FIG. 4 represents the configuration of the emotion recognition device 101 in the case of receiving a biological information pattern variation amount and emotion information in the learning phase.

In the learning phase, the receiving unit 116 receives emotion information representing an emotion induced by a stimulus applied to a test subject, and the relative value of a biological information pattern obtained by applying the stimulus to the test subject. In the learning phase, the receiving unit 116 associates the relative value of the biological information pattern with the emotion represented by the received emotion information. The receiving unit 116 stores the relative value of the biological information pattern, associated with the emotion, for example, in the measured data storage unit 117. Alternatively, the receiving unit 116 may send the relative value of the biological information pattern, associated with the emotion, to the classification unit 110.

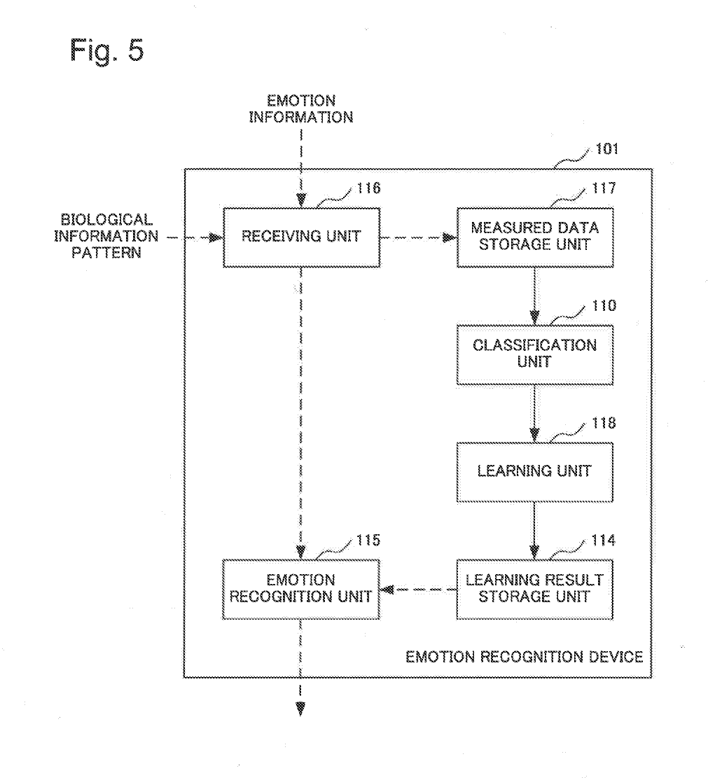

FIG. 5 is a second block diagram representing an example of a configuration of the emotion recognition device 101 in a learning phase. FIG. 5 represents the configuration of the emotion recognition device 101 in the case of carrying out supervised machine learning based on a biological information pattern variation amount associated with emotion information in the learning phase.

The classification unit 110 classifies, as described below, a relative value of biological information pattern stored in the measured data storage unit 117 into a group of the relative values of biological information patterns which are associated with emotions belonging to the same emotion class.

In the present comparative example and each exemplary embodiment of the present invention, an emotion input by an experimenter is selected from, for example, a plurality of emotions determined in advance. In other words, the emotions associated with the relative values of the biological information patterns are emotions selected from the plurality of emotions determined in advance.

An emotion in the plurality of emotions is characterized by, for example, one or more classes of emotions, to which the emotion belongs. In the description of the comparative example and each exemplary embodiment of the present invention, a class of an emotion is also referred to simply as “class”. The group of one or more classes is also referred to as “emotion class”. The emotion class is, for example, the set of the states of emotions classified depending on features. For example, the state of an emotion is classified into one of classes for one axis. The axis represents, for example, a viewpoint for evaluating a feature of a state of an emotion. The state of an emotion in each axis may be classified independently from the other axes. In the following description, a class classified in one axis is also referred to as “base class”. The base class is one of emotion classes. The product set of base classes in different plural axes is also one of the emotion classes.

In the present comparative example and each exemplary embodiment of the present invention, each of the emotions is, for example, a product set of base classes in all defined axes. Accordingly, an emotion in the plurality of emotions is represented by all the base classes to which the emotion belongs. An emotion in the plurality of emotions is also an emotion class. The emotion of a test subject (i.e., an emotion including a state of an emotion of a test subject) is specified by specifying base classes including the state of the emotion of the test subject in all the defined axes. In a case where the result of evaluation of the feature of the state of an emotion is represented as a numerical value in each axis, the axis corresponds to a coordinate axis. In this case, an origin represents the emotion of a test subject at rest.

In the comparative example and each exemplary embodiment of the present invention below, an example in which the number of axes is two is described as a specific example. The two axes are represented by α and β. The number of classes per axis is two. Classes for the axis α (i.e., base classes of axis α) are α1 and α2. Classes for the axis 13 (i.e., base classes of axis β) are β1 and β2. Each emotion is classified into α1 or α2. Further, each emotion is classified into β1 or β2 independently from the classification into α1 or α2. In other words, each emotion is included in α1 or α2. Further, each emotion is also included in β1 or β2. Each emotion is specified by the class for the axis α including the emotion and the class for the axis β including the emotion. In other words, each emotion can be represented by classes for an axis α and classes for an axis β. In this case, four emotions can be represented by those classes. The axes and classes of emotions may be predetermined by, for example, a constructor of a system, or an experimenter.

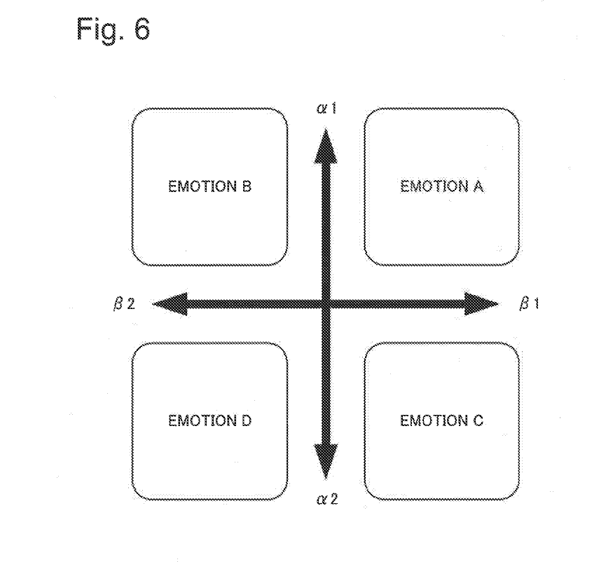

FIG. 6 is a view representing an example of the classified emotions. In FIG. 6, the vertical axis corresponds to the axis α. The emotions in the upper half are classified into the class α1. The emotions in the lower half are classified into the class α2. The horizontal axis corresponds to the axis β. The emotions in the right half are classified into the class β1. The emotions in the left half are classified into the class β2. In the example illustrated in FIG. 6, the emotion A is included in α1 and β1. The emotion B is included in α1 and β2. The emotion C is included in α2 and β1. The emotion D is included in α2 and β2. As described above, an emotion is represented by classes including the emotion. For example, the emotion A is represented by α1 and β1.

The classification unit 110 selects one emotion class from, for example, a plurality of emotion classes determined in advance. This emotion class is, for example, an emotion class determined by one or more base classes. The plurality of classes may be all the base classes that are defined. The plurality of emotion classes may be all the emotions that are defined. The classification unit 110 extracts all the relative values of biological information patterns, associated with emotions included in the selected emotion class, for example, from the relative values of biological information patterns stored in the measured data storage unit 117. The classification unit 110 may repeat the selection of the emotion class and the extraction of the relative values of the biological information patterns, associated with the emotions included in the selected emotion class, until completion of the selection from the plurality of emotion classes determined in advance. The classification unit 110 may select the same relative values of biological information patterns several times.

As described above, the classification unit 110 classifies the relative values of the biological information patterns stored in the measured data storage unit 117 into the groups of the relative values of the biological information patterns, associated with the emotions belonging to the same emotion classes. One relative value of a biological information pattern may be included in plural groups. In the following description, “emotion class associated with group” refers to an emotion class to which emotions associated with the relative values of biological information patterns included in the group belong.

The classification unit 110 may send an emotion class and the extracted relative value of biological information patterns to the learning unit 118, for example, for each of the emotion classes. In other words, the classification unit 110 may send an emotion class associated with a group, and the relative values of biological information patterns included in the group to the learning unit 118 for each of the groups. The classification unit 110 may send, for example, a class identifier by which the selected emotion class is specified, and the selected relative values of biological information patterns to the learning unit 118.

In the example illustrated in FIG. 6, the classification unit 110 may sequentially select one emotion, for example, from the emotion A, the emotion B, the emotion C, and the emotion D. In this case, the classification unit 110 may select the relative values of biological information patterns associated with the selected emotion. The classification unit 110 may sequentially select one class, for example, from α1, α2, β1, and β2. In this case, the classification unit 110 may select the relative values of biological information patterns associated with an emotion belonging to the selected class. For example, when α1 is selected, the classification unit 110 may select the relative values of the biological information patterns associated with the emotion A or the emotion B.

The learning unit 118 carries out learning by a supervised machine learning method based on the received classes and on the received relative values of the biological information patterns. The learning unit 118 stores the result of the learning in the learning result storage unit 114. The learning unit 118 may derive probability density distributions of the received emotion classes, for example, based on the received emotion classes and on the received relative values of the biological information patterns. The learning unit 118 may store the probability density distributions of the received classes as a learning result in association with the emotion class in the learning result storage unit 114.

In a case where the number of feature quantities extracted by the biological information processing unit 221 is d, the relative values of biological information patterns are represented by vectors in a d-dimensional feature space. The learning unit 118 plots vectors represented by the received relative values of the biological information patterns, in the d-dimensional feature space, separately for each of the emotion classes associated with the relative values of the biological information patterns, so that the origin is initial points of the vectors. The learning unit 118 estimates the probability density distribution of the vectors represented by the relative values of the biological information patterns for each of the emotion classes based on the distribution of terminal points of the vectors plotted in the d-dimensional feature space. As described above, the emotion class associated with the relative values of the biological information patterns received by the learning unit 118 is, for example, an emotion. The emotion class associated with the relative values of the biological information patterns received by the learning unit 118 may be, for example, a base class.

In a case where the emotion class associated with the relative values of the biological information patterns received by the learning unit 118 is an emotion, the learning unit 118 selects one emotion from all the emotions determined in advance. When the selected emotion is, for example, an emotion A, the learning unit 118 generates, in the d-dimensional feature space, the distribution of the terminal points of vectors representing the relative values of the biological information patterns associated with the emotion A. The relative values of the biological information patterns associated with the emotion A represent changes in feature quantities when the state of a test subject changes from a state in which the test subject is at rest to a state in which the emotion A is induced. The distribution, in the d-dimensional feature space, of the terminal points of the vectors representing the relative values of the biological information patterns associated with the emotion A is the distribution of the changes in the feature quantities when the state of the test subject changes from the state in which the test subject is at rest to the state in which the emotion A is induced.

The learning unit 118 further estimates, based on the generated distribution, the probability density distribution of the relative values of the biological information patterns when the state of the test subject changes from the state in which the test subject is at rest to the state in which the emotion A is induced. In the following description of the present comparative example, the probability density distribution of the relative values of the biological information patterns when the state of the test subject changes from the state in which the test subject is at rest to the state in which the emotion A is induced is referred to as “probability density distribution of emotion A”.

The learning unit 118 stores the probability density distribution of emotion A in the learning result storage unit 114. Various forms are possible as the form of the probability density distribution stored in the learning result storage unit 114 as long as a d-dimensional vector and a probability are associated with each other in the form. For example, the learning unit 118 divides the d-dimensional feature space into meshes having predetermined sizes and calculates the probability for each of the meshes. The learning unit 118 may store the calculated probability associated with an identifier of a mesh and an emotion in the learning result storage unit 114.

The learning unit 118 repeats generation of a distribution and estimation of a probability density distribution based on the distribution while sequentially selecting one emotion, for example, until all the emotions determined in advance are selected. For example, when the emotion B, the emotion C, and the emotion D are present in addition to the emotion A, the learning unit 118 sequentially estimates the probability density distributions of the emotion B, the emotion C, and the emotion D in a manner similar to that in the estimation of the probability density distribution of the emotion A. The learning unit 118 stores the estimated probability density distributions associated with emotions in the learning result storage unit 114.

If the probability density distribution of each emotion is estimated with high accuracy, the emotion of a test subject can be estimated with high accuracy in the case of measuring a biological information data associated with an unknown emotion (referred to as “test data”). In the following description, a case in which biological information data is biological information data measured when a stimulus for inducing an emotion X is applied to a test subject is referred to as “biological information data belongs to emotion X”. A case in which a relative value of a biological information pattern is the relative value of the biological information pattern derived from biological information data measured when a stimulus for inducing an emotion X is applied to a test subject is referred to as “relative value of biological information pattern belongs to emotion X”. Further, a case in which a feature vector x represents the relative value of a biological information pattern derived from biological information data measured when an emotion induced by a test subject is an emotion X is referred to as “feature vector x belongs to emotion X”.

The emotion recognition system 201 of the present comparative example in an estimation phase is described next.

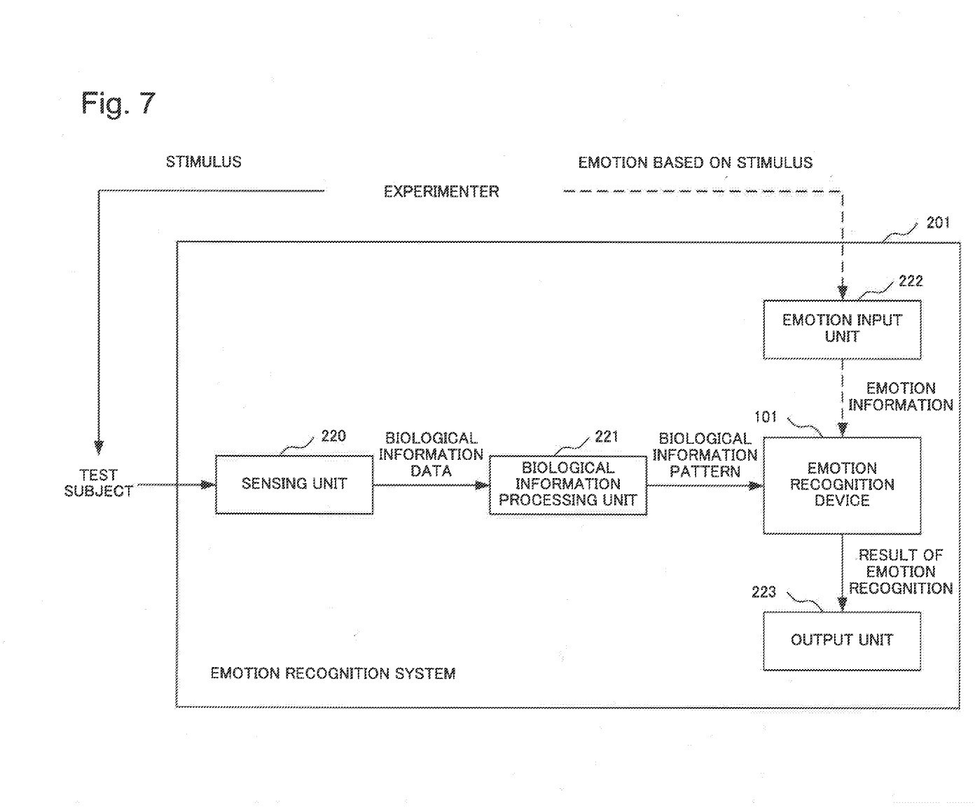

FIG. 7 is a block diagram representing an example of a configuration of the emotion recognition system 201 in the estimation phase. According to FIG. 7, an experimenter applies a stimulus to a test subject who is in the state of being at rest also in the estimation phase. In the estimation phase, the sensing unit 220 and the biological information processing unit 221 operate in a manner similar to that in the learning phase. The sensing unit 220 measures the biological information of a test subject of which the state changes from a state in which the test subject is at rest to a state in which an emotion is induced by a stimulus. The biological information processing unit 221 receives, from the sensing unit 220, biological information data representing the result of measurement of the biological information. The biological information processing unit 221 extracts a resting pattern and a stimulation pattern from the received biological information data in a manner similar to that in the learning phase. The biological information processing unit 221 sends the relative values of the biological information patterns to the emotion recognition device 101.

In the estimation phase, the experimenter does not input any emotion. In the estimation phase, the emotion input unit 222 does not send any emotion information to the emotion recognition device 101.

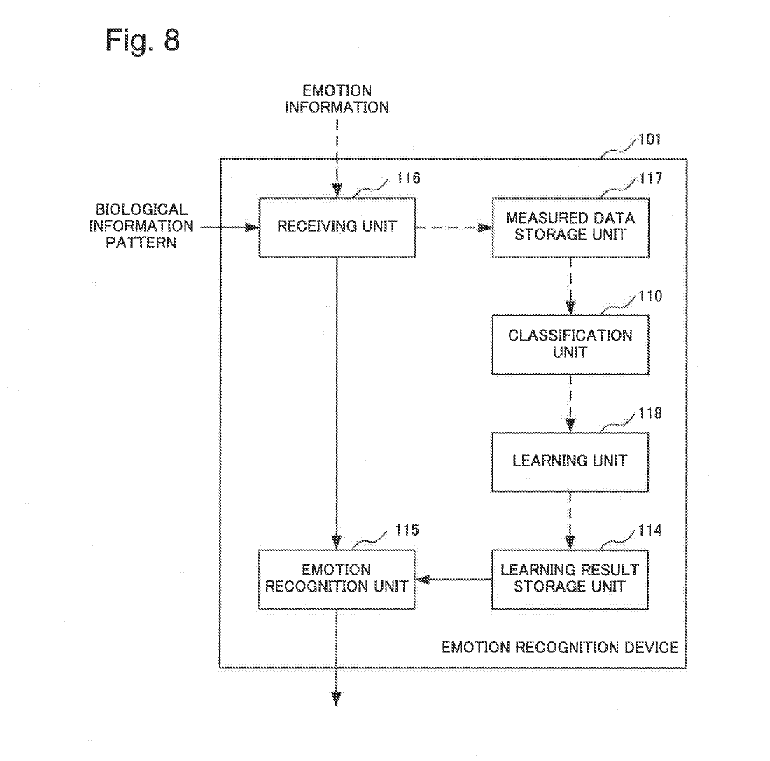

FIG. 8 is a block diagram representing an example of a configuration of the emotion recognition device 101 in an estimation phase. According to FIG. 8, the receiving unit 116 receives the relative values of biological information patterns. In the estimation phase, the receiving unit 116 does not receive any emotion information. In the estimation phase, the receiving unit 116 sends the relative values of the biological information patterns to the emotion recognition unit 115. The receiving unit 116 may carry out operation in the estimation phase (i.e., sending of biological information patterns to the emotion recognition unit 115) when receiving only the relative values of the biological information patterns and not receiving any emotion information. The receiving unit 116 may carry out operation in a learning phase (i.e., storing relative values of biological information patterns in the measured data storage unit 117) when receiving the relative values of the biological information patterns and emotion information.

The emotion recognition unit 115 estimates, based on the learning result stored in the learning result storage unit 114, an emotion induced in the test subject at the time when measuring biological information data from which the received relative values of the biological information patterns are derived.

Specifically, the emotion recognition unit 115 estimates the emotion, for example, based on a calculation method described below. As described above, in the description of the present comparative example and each exemplary embodiment of the present invention, the relative value of a biological information pattern is also referred to as a “biological information pattern variation amount”, in the description of the present comparative example and each exemplary embodiment of the present invention.

In the following description, a vector x represents a feature vector which is a vector representing a biological information pattern variation amount. A probability density function p(x|ωᵢ) indicating a probability density distribution that a feature vector x belongs to an emotion ωᵢ, which is estimated in a learning phase, represents a probability density function indicating a probability density distribution that x belongs to the emotion ωᵢ. As described above, the probability density distribution that the feature vector x belongs to the emotion ωᵢ is estimated for each i in the learning phase. The probability P(ωᵢ) represents the occurrence probability of the emotion ωᵢ. Further, a probability P(ωᵢ|x) represents a probability that an emotion to which x belongs is ωᵢ when x is measured. In this case, an equation shown in Math. 1 hold true according to Bayes' theorem.

$$P ( {ω}_{i} \vert x ) = \frac{p ( x \vert {ω}_{i} )}{{\sum }_{i = 1}^{C} P ( {ω}_{i} ) p ( x \vert {ω}_{i} )} P ( {ω}_{i} ) \qquad [ Math . 1 ]$$

By using the expression shown in Math. 1, a probability that the feature vector x obtained in the estimation phase belongs to each of the emotions is determined based on an emotion identification map, i.e., p(x|ωᵢ), determined by learning data, and on the occurrence probability P(ωᵢ) of the emotion. As described above, the accuracy of emotion recognition depends on the accuracy of estimation of the probability density distribution of each of the emotions in the feature space of biological information.

For example, a linear discrimination method can be adopted as a method (discriminator) of estimating a probability density distribution p(x|ωᵢ). When four emotions targeted for estimation are present, the emotions can be identified by repeating two-class classification two times. When an emotion is identified using a linear discrimination method and two-class classification carried out multiple times, it is preferable to first convert a d-dimensional feature space (d is the number of extracted feature quantities) into an optimal one-dimensional space in order to carry out the first two-class classification. A within-class covariance matrix Σ_{W} and a between-class covariance matrix Σ_{B} of two classes (for example, class α1 and class α2) in the first two-class classification are defined as shown in the following equations.

$$\begin{matrix}\begin{matrix}{\sum }_{W} ≡ {\sum }_{i = α 1 , α 2} P ( {ω}_{i} ) {\sum }_{i} \\ = {\sum }_{i = α 1 , α 2} ( P ( {ω}_{i} ) \frac{1}{{n}_{i}} {\sum }_{x ∈ {χ}_{i}} ( x - {m}_{i} ) {( x - {m}_{i} )}^{t} )\end{matrix} & [ Math . 2 ] \\ \begin{matrix}{\sum }_{B} ≡ {\sum }_{i = α 1 , α 2} P ( {ω}_{i} ) ( {m}_{i} - m ) {( {m}_{i} - m )}^{t} \\ = {\sum }_{i = α 1 , α 2} \frac{{n}_{i}}{n} ( {m}_{i} - m ) {( {m}_{i} - m )}^{t} \\ = \frac{{n}_{α 1}}{n} ( {m}_{α 1} - \frac{{n}_{α 1} {m}_{α 1} + {n}_{α 2} {m}_{α 2}}{n} ) \\ {( {m}_{α 1} - \frac{{n}_{α 1} {m}_{α 1} + {n}_{α 2} {m}_{α 2}}{n} )}^{t} + \\ \frac{{n}_{α 2}}{n} ( {m}_{α 2} - \frac{{n}_{α 1} {m}_{α 1} + {n}_{α 2} {m}_{α 2}}{n} ) \\ {( {m}_{α 2} - \frac{{n}_{α 1} {m}_{α 1} + {n}_{α 2} {m}_{α 2}}{n} )}^{t} \\ = P ( {ω}_{α 1} ) P ( {ω}_{α 2} ) ( {m}_{α 1} - {m}_{α 2} ) {( {m}_{α 1} - {m}_{α 2} )}^{t}\end{matrix} & [ Math . 3 ]\end{matrix}$$

The vector m represents the mean vector of all feature vectors, and the vector mᵢ (i=α1, α2) represents the mean vector of feature vectors belonging to each of the classes. The integer n represents the number of all the feature vectors, and the integer nᵢ represents the number of the feature vectors belonging to each of the classes. Further, the set χᵢ represents the set of all the feature vectors belonging to each of the classes.

Equations shown in Math. 4, Math. 5, and Math. 6 hold true according to those definitions.

$$\begin{matrix}n = {n}_{α 1} + {n}_{α 2} & [ Math . 4 ] \\ m = \frac{{n}_{α 1} {m}_{α 1} + {n}_{α 2} {m}_{α 2}}{n} & [ Math . 5 ] \\ P ( {ω}_{i} ) = \frac{{n}_{i}}{n} , ( i = α 1 , α 2 ) & [ Math . 6 ]\end{matrix}$$

In the deformation of the equation shown in Math. 3, the relations shown in Math. 4, Math. 5, and Math. 6 are used.

Σᵢ shown in Math. 7 is the covariance matrix of feature vectors belonging to each class i.

$${\sum }_{i} ≡ \frac{1}{{n}_{i}} {\sum }_{x ∈ {χ}_{i}} ( x - {m}_{i} ) {( x - {m}_{i} )}^{t} \qquad [ Math . 7 ]$$

The dimension of a feature space is d which is the number of feature quantities that are extracted. Accordingly, a matrix A representing conversion from the feature space into a one-dimensional space is a (d, 1) matrix (matrix with d rows and one column) representing conversion from a d-dimensional feature space into the one-dimensional space. A function J_{Σ}(A) representing a degree of separation between classes by A is defined by an expression shown in Math. 8. The emotion recognition unit 115 determines a transformation matrix A that maximizes the function J_{Σ}.

$${J}_{Σ} ( A ) = \frac{{A}^{t} {Σ}_{B} A}{{A}^{t} {Σ}_{W} A} \qquad [ Math . 8 ]$$

Math. 9 represents a probability density distribution on a one-dimensional axis, defined using the transformation matrix A.

$$p ( x \vert {ω}_{α 1} ) = \{ \begin{matrix}1 , & ( \vert {A}^{t} x - {A}^{t} {m}_{α 1} \vert - \vert {A}^{t} x - {A}^{t} {m}_{α 2} \vert \geq 0 ) \\ 0 , & ( \vert {A}^{t} x - {A}^{t} {m}_{α 1} \vert - \vert {A}^{t} x - {A}^{t} {m}_{α 2} \vert < 0 )\end{matrix} \\ p ( x \vert {ω}_{α 2} ) = \{ \begin{matrix}0 , & ( \vert {A}^{t} x - {A}^{t} {m}_{α 1} \vert - \vert {A}^{t} x - {A}^{t} {m}_{α 2} \vert \geq 0 ) \\ 1 , & ( \vert {A}^{t} x - {A}^{t} {m}_{α 1} \vert - \vert {A}^{t} x - {A}^{t} {m}_{α 2} \vert < 0 )\end{matrix} \qquad [ Math . 9 ]$$

The equations shown in Math. 9 represents the definition of probability density distributions, which users a centroid (i.e. mean vector) of each of the classes as a prototype for class identification. Such probability density distributions may be defined using feature vectors in the vicinities of class boundaries as prototypes depending on data obtained in a learning phase.

In an estimation phase, the emotion recognition unit 115 estimates a probability that an obtained feature vector belongs to a class, for each of the classes, based on a probability obtained by substituting the probability density distribution described above into the equation shown in Math. 1. The emotion recognition unit 115 may determine that the feature vector belongs to a class of which the estimated probability is high.

In a similar manner, the emotion recognition unit 115 further determines whether the feature vector belongs to either of the two next classes (for example, class β1 and class β2). As thus described, the emotion recognition unit 115 determines which of the four classes of the emotion A (α1 and β1), the emotion B (α1 and β2), the emotion C (α2 and β1), and the emotion D (α2 and β2) the feature vector belongs to.

In the present comparative example, a test subject in the estimation phase is not limited to a particular test subject.

The operation of the emotion recognition system 201 of the present comparative example is described next in detail with reference to the drawings. The operation of the emotion recognition system 201 excluding the emotion recognition device 101, and the operation of the emotion recognition device 101 are separately described below.

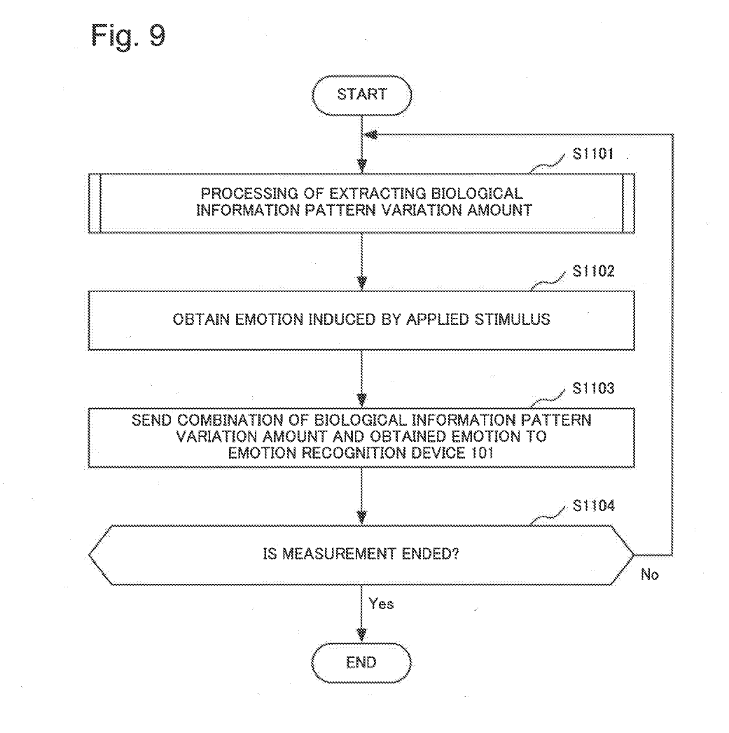

FIG. 9 is a flowchart representing an example of an operation of the emotion recognition system 201 in a learning phase.

According to FIG. 9, first, the emotion recognition system 201 carries out processing of extracting a biological information pattern variation amount by the sensing unit 220 and the biological information processing unit 221 (step S1101). “Processing of extracting a biological information pattern variation amount” represents processing of acquiring biological information data by measurement and deriving the biological information pattern variation amount from the acquired biological information data. A test subject from whom the biological information pattern variation amount is acquired in step S1101 is not limited to a particular test subject. The processing in step S1101 is described later.

An experimenter inputs, by the emotion input unit 222, an emotion induced by a stimulus applied to the test subject by the experimenter in step S1101. The emotion input unit 222 obtains the emotion input by the experimenter (step S1102).

The biological information processing unit 221 sends the derived biological information pattern variation amount to the emotion recognition device 101. The emotion input unit 222 sends the emotion input by the experimenter to the emotion recognition device 101. In other words, the emotion recognition system 201 sends the combination of the biological information pattern variation amount and the emotion to the emotion recognition device 101 (step S1103).

When the measurement of the biological information is not ended (No in step S1104), the emotion recognition system 201 repeats the operations from step S1101 to step S1103. In step S1101, the experimenter may carry out arrangement, for example, such that the emotion recognition system 201 measures the biological information of a number of different test subjects while varying stimuli applied to test subjects. When the measurement is ended (Yes in step S1104), the emotion recognition system 201 ends the operation of the learning phase. In step S1104, the emotion recognition system 201 may determine that the measurement of the biological information is ended, for example, when the experimenter directs the emotion recognition system 201 to end the measurement. The emotion recognition system 201 may determine that the measurement of the biological information is not ended, for example, when the experimenter directs the emotion recognition system 201 to continue the measurement.

The operation of the processing of extracting a biological information pattern variation amount by the emotion recognition system 201 is described next in detail with reference to the drawings.

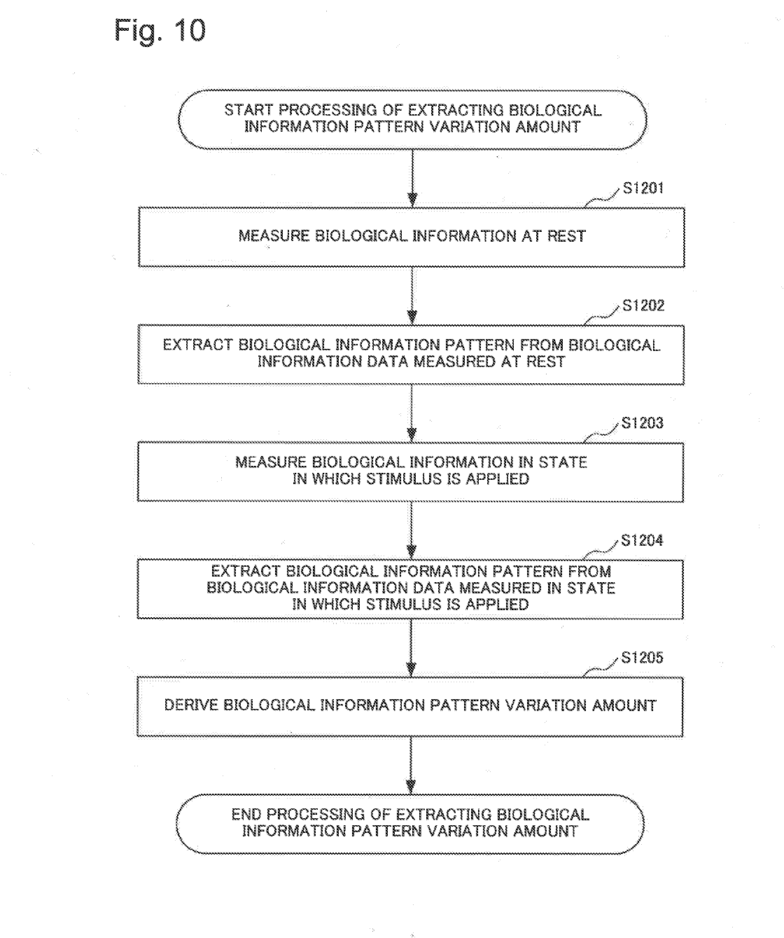

FIG. 10 is a flowchart representing an example of the operation of the processing of extracting aa biological information pattern variation amount by the emotion recognition system 201.

According to FIG. 10, the sensing unit 220 measures the biological information of a test subject at rest (step S1201). The sensing unit 220 sends, to the biological information processing unit 221, the biological information data obtained by the measurement. The biological information processing unit 221 extracts a biological information pattern from the biological information data measured at rest (step S1202). The sensing unit 220 measures the biological information of a test subject to which a stimulus is applied (step S1203). The sensing unit 220 sends, to the biological information processing unit 221, the biological information data obtained by the measurement. The biological information processing unit 221 extracts a biological information pattern from the biological information data measured in the state where a stimulus is applied (step S1204). The biological information processing unit 221 derives the variation amount between the biological information pattern at rest and the biological information pattern in the state where a stimulus is applied (step S1205).

In the example represented in FIG. 10, the biological information processing unit 221 may specify biological information data at rest and biological information data in the state where a stimulus is applied, for example, based on instructions from an experimenter. The biological information processing unit 221 may specify the biological information data at rest and the biological information data in the state where a stimulus based on a time period elapsed from the start of the measurement, and on magnitude of a change in biological information data.

The biological information processing unit 221 may specify, as the biological information data in the state where a stimulus is applied, for example, biological information data measured after the time period elapsed from the start of the measurement exceeds a predetermined time period. The biological information processing unit 221 may determine that a stimulus starts to be applied, for example, when magnitude of a change between the measured biological information data and biological information data measured at the time of the start of the measurement or at the time of a lapse of predetermined time period since the start of the measurement exceeds a predetermined value. The predetermined time is, for example, an amount of experimentally derived time, at rest, from the start of the measurement until the time when the biological information data becomes stabilized. The biological information processing unit 221 may specify, for example, biological information data measured after determining that the stimulus starts to be applied as the biological information data in the state where the stimulus is applied.

The operation of the emotion recognition device 101 in a learning phase is described next in detail with reference to the drawings.

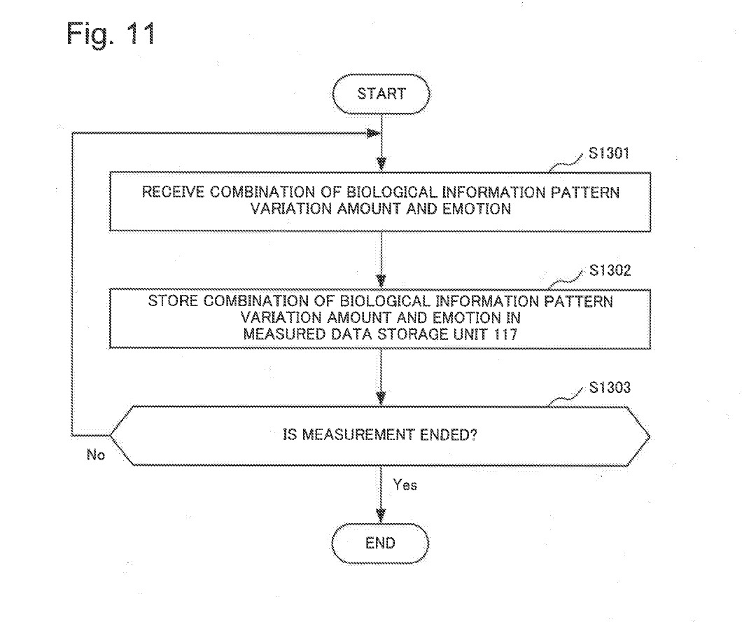

FIG. 11 is a flowchart representing an example of a first operation of the emotion recognition device 101 in the learning phase. FIG. 11 represents the operation of the emotion recognition device 101 during manipulation of measuring the biological information of a test subject by an experimenter.

The receiving unit 116 receives a combination of a biological information pattern variation amount and an emotion (step S1301). The biological information pattern variation amount and the emotion received by the receiving unit 116 in step S1301 are the biological information pattern variation amount and the emotion sent to the emotion recognition device 101 in step S1103. In a learning phase, the receiving unit 116 associates the received biological information pattern variation amount and the emotion with each other, and stores the biological information pattern variation amount and the emotion associated with each other in the measured data storage unit 117 (step S1302). When the measurement is not ended (No in step S1303), the emotion recognition device 101 repeats the operations of step S1301 and step S1302. When the measurement is ended (Yes in step S1303), the emotion recognition device 101 ends the operation represented in FIG. 11. The emotion recognition device 101 may determine whether the measurement is completed or not, for example, based on instructions from an experimenter.

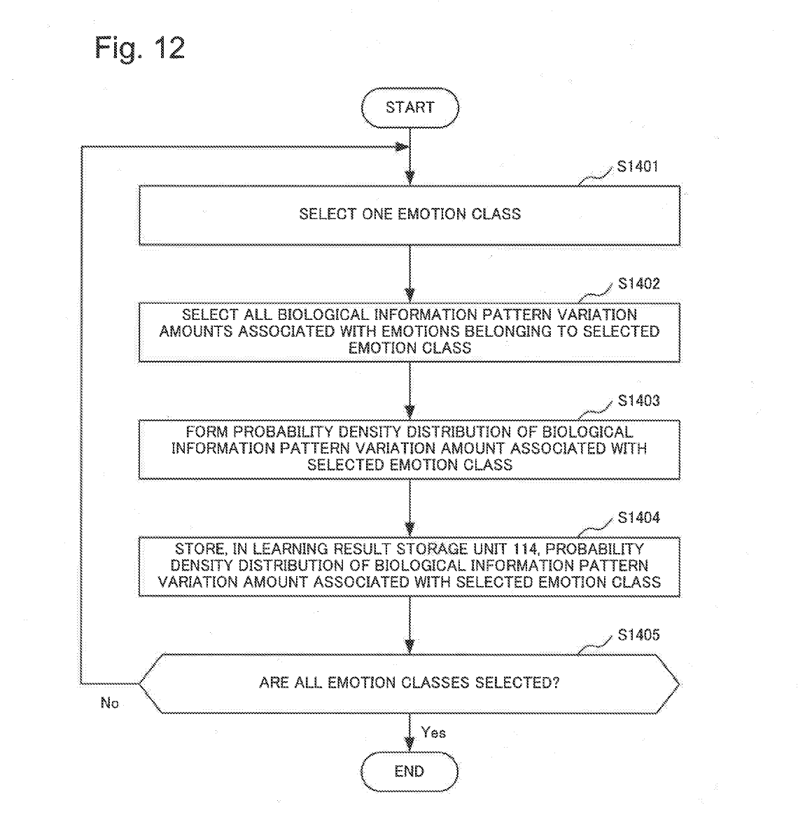

FIG. 12 is a flowchart representing an example of the second operation of the emotion recognition device 101 in a learning phase. FIG. 12 represents the operation of the emotion recognition device 101 carrying out learning based on supervised machine learning by using a biological information pattern variation amount and an emotion associated with the variation amount.

The classification unit 110 selects one emotion class from a plurality of emotion classes determined in advance (step S1401). As described above, the emotion classes are, for example, emotions determined in advance. The emotion classes may be, for example, the above-described base classes determined in advance. The classification unit 110 selects all biological information pattern variation amounts associated with emotions included in the selected emotion class (step S1402). The learning unit 118 forms the probability density distribution of the biological information pattern variation amounts belonging to the selected emotion class (step S1403). The learning unit 118 stores the formed probability density distribution, associated with the selected emotion class, in the learning result storage unit 114 (step S1404). When any emotion class that is not selected exists (No in step S1405), the emotion recognition device 101 repeats the operations of from step S1401 to step S1404. When all the emotion classes are selected (Yes in step S1405), the emotion recognition device 101 ends the operation represented in FIG. 12.

The operation of the emotion recognition system 201 in a determination phase is described next in detail with reference to the drawings.

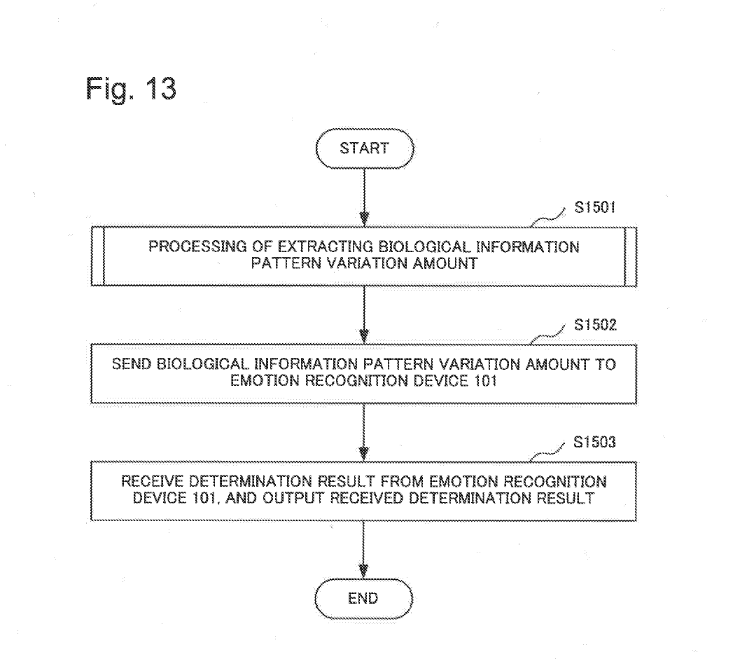

FIG. 13 is a flowchart representing an example of an operation of the emotion recognition system 201 in the determination phase. According to FIG. 13, first, the emotion recognition system 201 carries out processing of extracting a biological pattern variation amount in the determination phase (step S1501). In step S1501, the emotion recognition system 201 carries out the operation represented in FIG. 10. As described above, the processing of extracting a biological information pattern variation amount is processing of acquiring biological information data and deriving a biological information pattern variation amount from the acquired biological information data. Then, the biological information processing unit 221 sends the biological information pattern variation amount to the emotion recognition device 101 (step S1502). The emotion recognition device 101, upon receiving the biological information pattern variation amount, estimates the emotion of a test subject, and sends a reply of the estimated emotion. The output unit 223 receives the emotion estimated by the emotion recognition device 101, and outputs the received emotion (step S1503).

The operation of the emotion recognition device 101 in the determination phase is described next in detail with reference to the drawings.

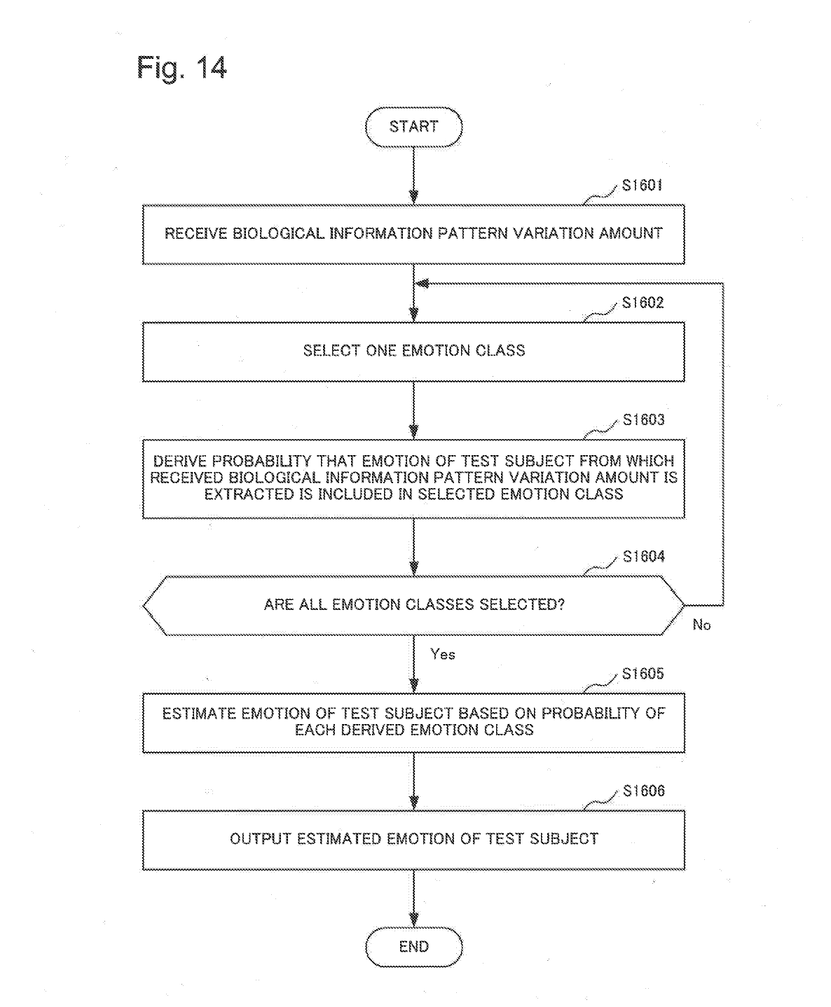

FIG. 14 is a drawing representing an example of an operation of the emotion recognition device 101 in the determination phase.

According to FIG. 14, the receiving unit 116 receives a biological information pattern variation amount from the biological information processing unit 221 (step S1601). In the determination phase, the receiving unit 116 does not receive any emotion. In the determination phase, the receiving unit 116 sends the received biological information pattern variation amount to the emotion recognition unit 115. The emotion determination unit 115 selects one emotion class from a plurality emotion classes determined in advance (step S1602). Using the probability density distribution stored in the learning result storage unit 114, the emotion recognition unit 115 derives a probability that the emotion of a test subject from which the received biological information pattern variation amount is extracted is included in the selected emotion class (step S1603). When any emotion class that is not selected exists (No in step S1604), the emotion recognition unit 115 repeats the operations of step S1602 and step S1603 until all the emotion classes are selected. When all the emotion classes are selected (Yes in step S1604), the emotion recognition unit 115 estimates the emotion of the test subject based on the derived probability of the emotion class (step S1605). As described above, the emotion classes are, for example, base classes. In this case, the emotion recognition unit 115 may estimate the emotion of the test subject by repeating two-class classification of selecting, from two base classes, a class including the emotion as described above. In other words, the emotion recognition unit 115 may select an emotion included in all the selected base classes as the emotion of the test subject. The emotion classes may be emotions. In this case, the emotion recognition unit 115 may select an emotion of which the derived probability is the highest as the emotion of the test subject. The emotion recognition unit 115 outputs the estimated emotion of the test subject (step S1606).

### First Exemplary Embodiment

An emotion recognition system 2 of a first exemplary embodiment of the present invention is described next in detail with reference to the drawings.

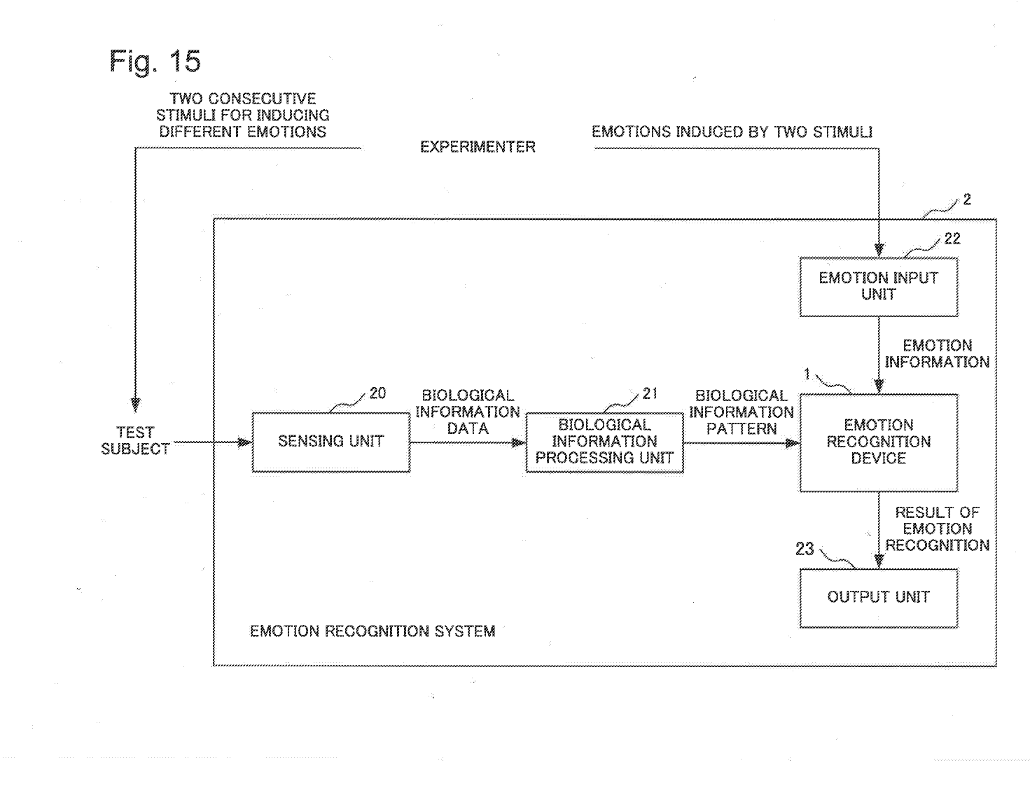

FIG. 15 is a block diagram representing an example of a configuration of the emotion recognition system 2 of the present exemplary embodiment. According to FIG. 15, the emotion recognition system 2 includes a sensing unit 20, a biological information processing unit 21, an emotion input unit 22, an emotion recognition device 1, and an output unit 23. In the example illustrated in FIG. 15, the emotion recognition system 2 is drawn as one device including the emotion recognition device 1. However, the emotion recognition system 2 may be implemented using plural devices. For example, the emotion recognition system 2 may be implemented using: a measuring device (not illustrated) including the sensing unit 20, the biological information processing unit 21, the emotion input unit 22, and output unit 23; and the emotion recognition device 1. In this case, the measuring device and the emotion recognition device 1 may be communicably connected to each other.

In the present exemplary embodiment, an experimenter separately applies two stimuli inducing different emotions to a test subject in one measurement of biological information. The emotions induced by the stimuli applied to the test subject are a combination of two emotions selected from a plurality of emotions determined in advance. The time periods for which the stimuli are applied are, for example, the quantity of time periods sufficient for inducing the emotions in the test subject, experimentally measured in advance. In the following description, a stimulus first applied by an experimenter in one measurement of biological information is also referred to as “first stimulus”. An emotion induced by the first stimulus is also referred to as “first emotion”. Similarly, a stimulus subsequently applied by the experimenter in one measurement of biological information is also referred to as “second stimulus”. An emotion induced by the second stimulus is also referred to as “second emotion”. The experimenter may start the measurement of the biological information of the test subject, for example, while applying the first stimulus to the test subject. The experimenter may vary the stimulus applied to the test subject to the second stimulus after the lapse of the above-described sufficient time from the start of the application of the first stimulus. The experimenter may end the measurement of the biological information of the test subject after the lapse of the above-described sufficient time from the start of the application of the second stimulus. As described above, the emotion of the test subject is expected to be varied from the first emotion to the second emotion by applying the stimulus to the test subject. In the case of varying the emotion of the test subject, the first emotion is an emotion before the variation, and the second emotion is an emotion after the variation.

The sensing unit 20 may include the same hardware configuration as that of the sensing unit 220 in the comparative example described above. The sensing unit 20 may operate in a manner similar to that of the sensing unit 220. The sensing unit 20 may be the same as the sensing unit 220 except the state of a test subject whose biological information is measured. The sensing unit 20 need not measure the biological information of the test subject at resting. The sensing unit 20 measures at least the biological information of the test subject to which the first stimulus is applied, and the biological information of the test subject to which the second stimulus is applied. The sensing unit 20 may start the measurement, for example, while the first stimulus is applied to the test subject. The sensing unit 20 may continue the measurement of the biological information until predetermined time has elapsed since the variation of the stimulus applied to the test subject to the second stimulus. The sensing unit 20 sends the biological information data obtained by the measurement to the biological information processing unit 21 in a manner similar to that of the sensing unit 220 in the comparative example.

The biological information processing unit 21 may have the same hardware configuration as that of the biological information processing unit 221 in the comparative example described above. The biological information processing unit 21 may carry out processing similar to that of the biological information processing unit 221. The biological information processing unit 21 may be the same as the biological information processing unit 221 except the state of a test subject whose biological information data from which a biological information pattern is derived is obtained by measurement. The biological information processing unit 21 extracts a biological information pattern (i.e., a first biological information pattern) from the biological information data obtained by the measurement in the state of receiving the first stimulus. The biological information processing unit 21 further extracts a biological information pattern (i.e., a second biological information pattern) from the biological information data obtained by the measurement in the state of receiving the second stimulus. A biological information pattern variation amount derived by the biological information processing unit 21 is a variation amount in the second biological information pattern with respect to the first biological information pattern. The biological information processing unit 21 derives the variation amount in the second biological information pattern with respect to the first biological information pattern.

The emotion input unit 22 may have the same hardware configuration as that of the emotion input unit 222 in the comparative example. An experimenter inputs emotions induced by two stimuli applied to a test subject, i.e., a first emotion and a second emotion to the emotion recognition system 2 through the emotion input unit 22. The emotion input unit 22 generates emotion information representing a change in emotion from the first emotion to the second emotion. The emotion input unit 22 inputs the generated emotion information into the emotion recognition device 1. The emotion information may be information capable of specifying that the change of the emotions induced in the test subject by the stimuli applied to the test subject by the experimenter is the change from the first emotion to the second emotion. The emotion information input into the emotion recognition device 1 by the emotion input unit 22 may include, for example, an emotion identifier of the first emotion and an emotion identifier of the second emotion. The emotion information input into the emotion recognition device 1 by the emotion input unit 22 may be associated with, for example, the emotion identifier of the first emotion and the emotion identifier of the second emotion.

The output unit 23 may have the same hardware configuration as that of the output unit 223 in the comparative example. The output unit 23 may operate in a manner similar to that of the output unit 223 in the comparative example.

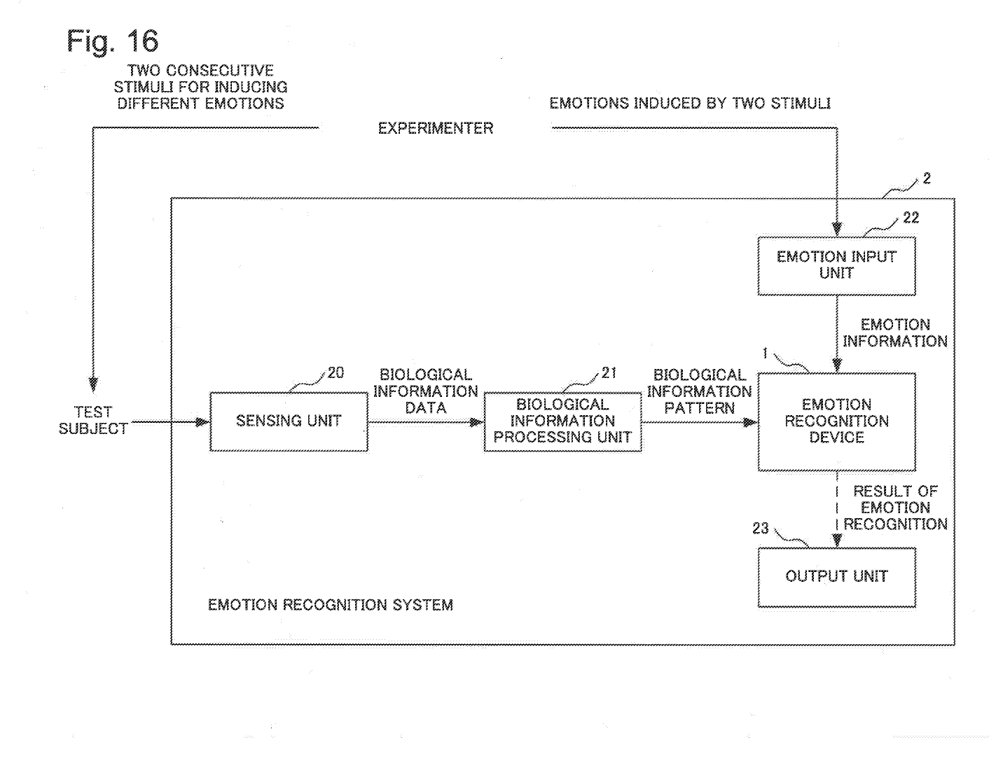

FIG. 16 is a block diagram representing an example of a configuration of the emotion recognition system 2 in a learning phase.

As described above, the sensing unit 20 measures the biological information of the test subject in a learning phase. The sensing unit 20 sends, to the biological information processing unit 21, the biological information data obtained by the measurement. The biological information processing unit 21 derives, from the received biological information data, a biological information pattern variation amount representing a change in the biological information of the test subject depended on a change in stimulus applied to the test subject. The biological information processing unit 21 sends the biological information pattern variation amount to the emotion recognition device 1. The emotion input unit 22 inputs, into the emotion recognition device 1, emotion information representing a change between emotions input by an experimenter. In the learning phase, the experimenter measures the biological information of the test subject, and inputs the emotion information representing the change in emotion from the first emotion to the second emotion, for example, while variously changing a combination of the first and second stimuli applied to the test subject. The test subject is not limited to a particular test subject. The experimenter may measure the biological information of an unspecified number of test subjects, and may input the emotion information of the test subjects. As a result, biological information pattern variation amounts and emotion information representing changes in emotion information are input by repetition into the emotion recognition device 1. The emotion recognition device 1 carries out learning in accordance with a supervised learning model by using the biological information pattern variation amounts and the emotion information representing the changes in the emotion information, which are input, as described below.

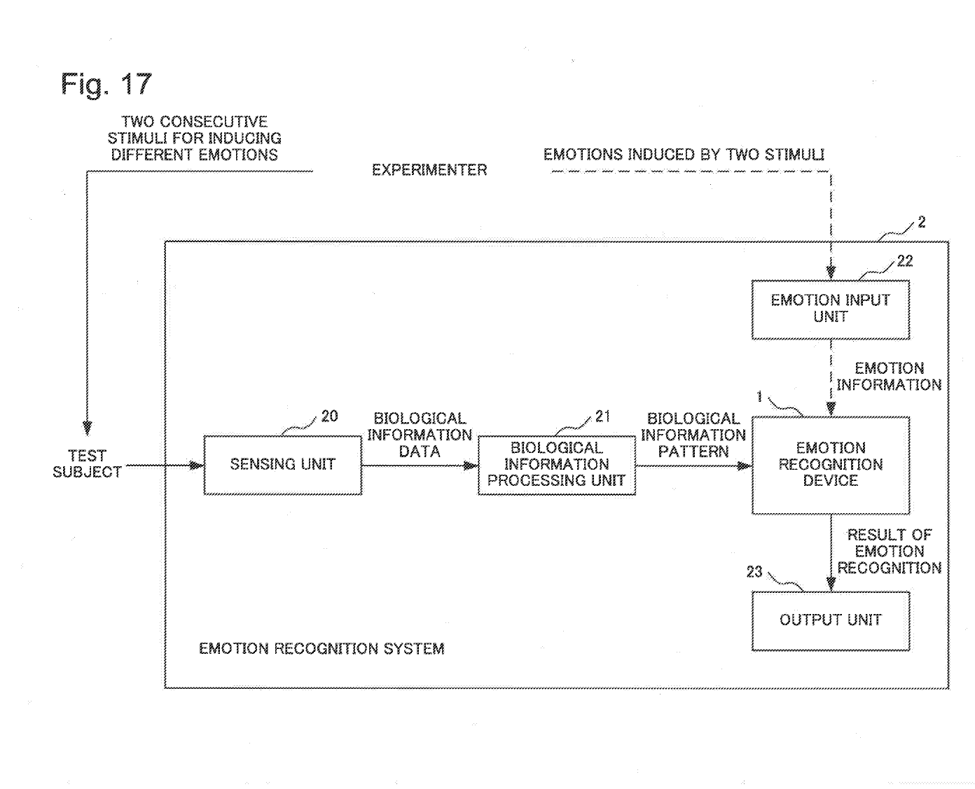

FIG. 17 is a block diagram representing an example of a configuration of the emotion recognition system 2 of the present exemplary embodiment in an estimation phase. In the estimation phase, an experimenter applies two consecutive stimuli for inducing different emotions to a test subject in a manner similar to that of the learning phase. Also in the estimation phase, the test subject is not limited to a particular test subject. In the estimation phase, the experimenter does not input any emotion information.

In the estimation phase, the sensing unit 20 and the biological information processing unit 21 operate in a manner similar to that of the learning phase. In other words, the sensing unit 20 sends, to the biological information processing unit 21, biological information data obtained by measurement. The biological information processing unit 21 sends, to the emotion recognition device 1, a biological information pattern variation amount extracted from the biological information data. The emotion input unit 22 does not input any emotion information into the emotion recognition device 1.

The emotion recognition device 1 estimates the emotion of the test subject based on the received biological information pattern variation amount as described below. The emotion recognition device 1 sends the estimated emotion of the test subject to the output unit 23. The emotion recognition device 1 may send, for example, an emotion identifier specifying the estimated emotion to the output unit 23.

The output unit 23 receives, from the emotion recognition device 1, the emotion estimated by the emotion recognition device 1 based on the biological information pattern variation amount input into the emotion recognition device 1 by the biological information processing unit 21. The output unit 23 may receive an emotion identifier specifying the estimated emotion. The output unit 23 outputs the received emotion. The output unit 23 may display, for example, a character string representing the emotion specified by the received emotion identifier. The method for outputting an emotion by the output unit 23 may be another method.

The emotion recognition device 1 of the present exemplary embodiment is described next in detail with reference to the drawings.

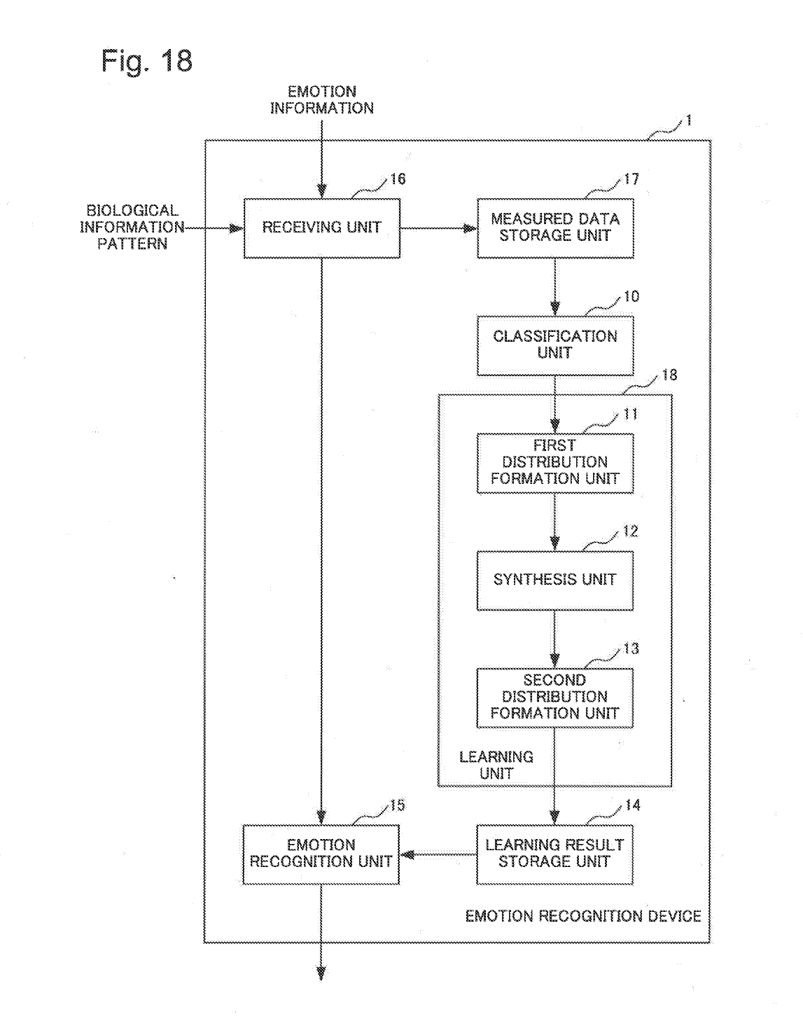

FIG. 18 is a block diagram representing an example of a configuration of the emotion recognition device 1 of the present exemplary embodiment. According to FIG. 18, the emotion recognition device 1 includes a receiving unit 16, a measured data storage unit 17, a classification unit 10, a learning unit 18, a learning result storage unit 14, and an emotion recognition unit 15. The learning unit 18 includes a first distribution formation unit 11, a synthesis unit 12, and a second distribution formation unit 13.

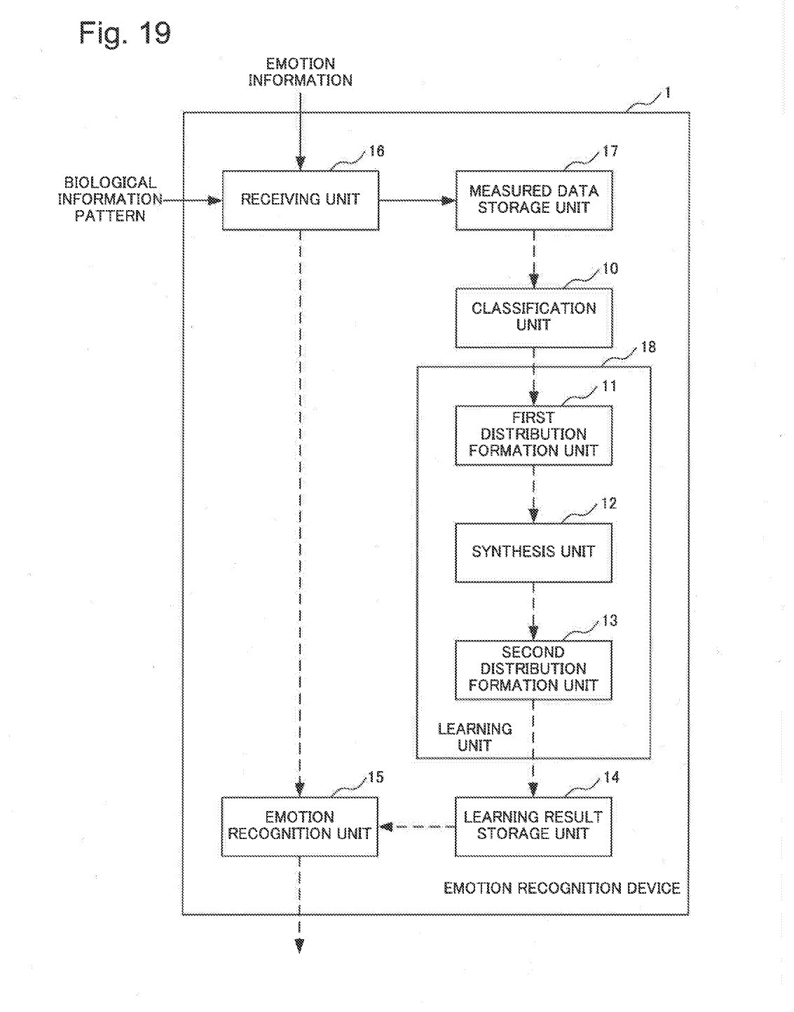

FIG. 19 is a first block diagram representing an example of a configuration of the emotion recognition device 1 in a learning phase. FIG. 19 represents the configuration of the emotion recognition device 1 in the case of receiving a biological information pattern variation amount and emotion information in the learning phase.

The receiving unit 16 receives the biological information pattern variation amount and the emotion information in the learning phase. As described above, the emotion information includes, for example, an identifier of a first emotion and an identifier of a second emotion. The receiving unit 16 receives biological information pattern variation amounts and emotion information by repetition based on measurement of the biological information and input of the emotion information by the experimenter. In the learning phase, the receiving unit 16 associates the received biological information pattern variation amounts and the emotion information with each other, and stores the biological information pattern variation amounts and the emotion information associated with each other in the measured data storage unit 17.

The biological information pattern variation amounts and the emotion information associated with each other are stored in the measured data storage unit 17. For example, a plurality of combinations of the biological information pattern variation amount and a piece of the emotion information associated with each other are stored in the measured data storage unit 17. As described above, in the present exemplary embodiment, the piece of the input emotion information is information capable of specifying the first emotion and the second emotion.

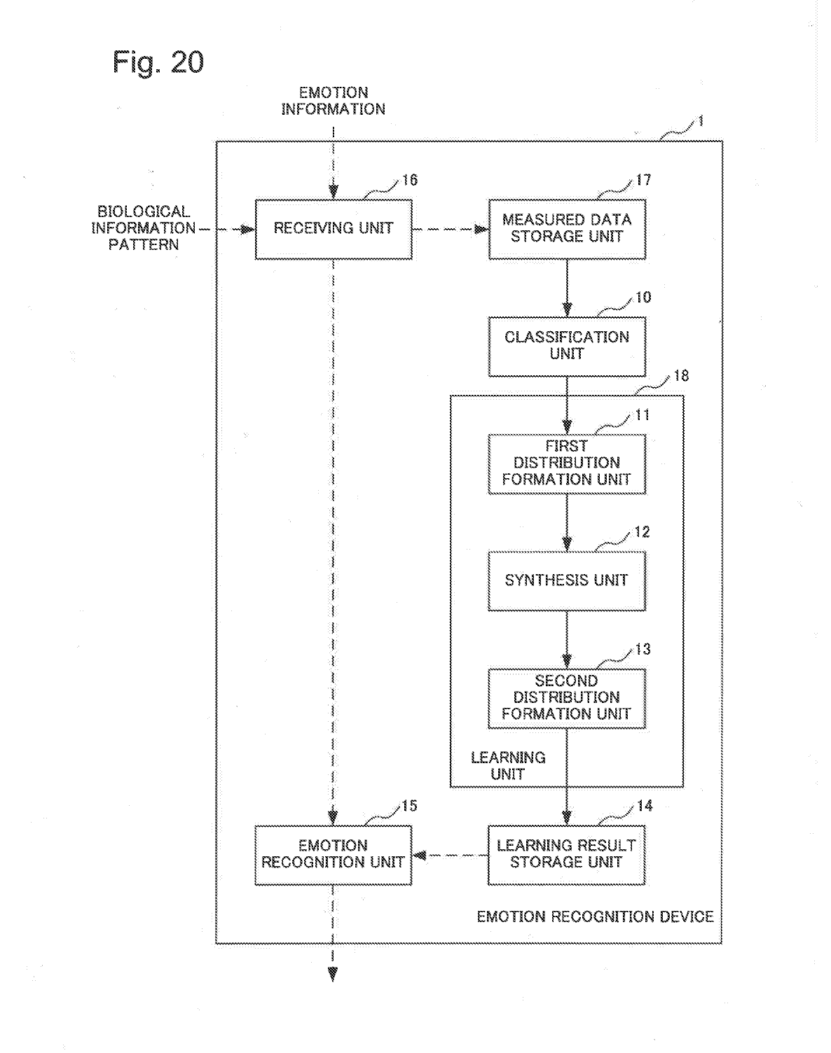

FIG. 20 is a second block diagram representing an example of the configuration of the emotion recognition device 1 in a learning phase. FIG. 20 represents the configuration of the emotion recognition device 1 in the case of carrying out learning based on combinations of the biological information pattern variation amounts and the emotion information associated with each other in the learning phase. The emotion recognition device 1 may carry out an operation in the learning phase, for example, according to instructions from an experimenter.

The classification unit 10 classifies the biological information pattern variation amounts, stored in the measured data storage unit 17, based on the emotion information associated with the biological information pattern variation amounts. In the present exemplary embodiment, a piece of the emotion information includes a first emotion and a second emotion as described above. The piece of the emotion information represents a change of emotion from the first emotion to the second emotion. The classification unit 10 may classify the biological information pattern variation amounts, for example, by generating groups of biological information pattern variation amounts associated with the same piece of the emotion information in the biological information pattern variation amounts stored in the measured data storage unit 17.

The learning unit 18 learns, based on the result of the classification of a biological information pattern variation amount by the classification unit 10, a relation between the biological information pattern variation amount and each of the above-described plurality of emotions as the second emotion in a case where the biological information pattern variation amount is obtained. The learning unit 18 stores the result of the learning in the learning result storage unit 14.

Specifically, first, the first distribution formation unit 11 forms a probability density distribution for each category, based on the result of the classification of the biological information pattern variation amounts stored in the measured data storage unit 17 by the classification unit 10. For example, when the biological information pattern variation amounts are classified according to pieces of the emotion information associated with the biological information pattern variation amounts, the first distribution formation unit 11 forms the probability density distribution for each change in emotion represented by the pieces of the emotion information.

The synthesis unit 12 synthesizes, for example, a plurality of groups having a common part in elements in the emotions after the changes, i.e., the second emotions, which are associated with the biological information pattern variation amounts, into one group.

In the second distribution formation unit 13, the second distribution formation unit 13 forms a probability density distribution for each group after the synthesis by the synthesis unit 12. The second distribution formation unit 13 stores, in the learning result storage unit 14, the probability density distribution formed for each group after the synthesis.

The results of the learning by the learning unit 18 are stored in the learning result storage unit 14. In other words, the results of the learning, stored by the second distribution formation unit 13, are stored in the learning result storage unit 14.

The emotion recognition device 1 in the learning phase is further specifically described below.

In the present exemplary embodiment, the plurality of emotions, axes, and classes on each of the axes are selected in advance so that an emotion is uniquely defined by all the classes to which the emotion belongs.

The emotion recognition device 1 in a case in which emotions are classified into two classes on each of two axes as described above is specifically described below. Like the example described above, the two axes are represented by α and β. Two classes on the axis α are referred to as “α1” and “α2”. Two classes on the axis β are referred to as “β1” and “β2”. Each of the emotions are classified into either α1 or α2 on the axis α. Each of the emotions are classified into either β1 or β2 on the axis β. Like the example described above, for example, an emotion A is an emotion belonging to both the classes α1 and β1. An emotion B is an emotion belonging to both the classes α1 and β2. An emotion C is an emotion belonging to both the classes α2 and β1. An emotion D is an emotion belonging to both the classes α2 and β2.

The classification unit 10 classifies, as described above, biological information pattern variation amounts stored in the measured data storage unit 17 such that the biological information pattern variation amounts having the same combination of a first emotion and a second emotion represented by emotion information associated therewith are included in the same group. For example, biological information pattern variation amounts obtained when an emotion induced by a stimulus is changed from the emotion A to the emotion B are classified into the same group.

The first distribution formation unit 11 forms a probability density distribution for each of the groups into which the biological information pattern variation amounts are classified. The probability density distribution generated by the first distribution formation unit 11 is represented by p(x|ωᵢ) described in the description of the comparative example. In the probability density distribution generated by the first distribution formation unit 11, ωᵢ is an emotion change. Like the description of the comparative example, x in this case is a biological information pattern variation amount.

The synthesis unit 12 synthesizes, as described above, a plurality of groups having a common part in elements in the emotions after the variations, i.e., the second emotions, which are associated with the biological information pattern variation amounts, into one group. The elements in a emotion are, for example, one or more classes to which the emotion belong. In the present exemplary embodiment, an emotion class is a group of one or more classes. For example, a method described below can be used as a method for synthesizing a plurality of groups by the synthesis unit 12 into one group.

In the following description, for example, the group of biological information pattern variation amounts associated with emotion information in which a first emotion is the emotion B and a second emotion is the emotion A is referred to as “a group of the emotion B to the emotion A”. The group of the biological information pattern variation amounts associated with the emotion information in which the first emotion is the emotion B and the second emotion is the emotion A is the group of biological information pattern variation amounts associated with emotion information representing a change from the emotion B to the emotion A.

For example, the synthesis unit 12 may synthesize, into one group, a plurality of groups of biological information pattern variation amounts associated with the emotion information in which all the classes to which the second emotions belong are common. In this case, groups in which the second emotions are the same are synthesized into one group. For example, the synthesis unit 12 synthesizes, into one group, groups in which second emotions are the emotion A, i.e., a group of the emotion B to the emotion A, a group of the emotion C to the emotion A, and a group of the emotion D to the emotion A. The group synthesized in this case is also referred to as “group to emotion A” in the following description. The synthesis unit 12 synthesizes, into one group, each of groups in which a second emotion is the emotion B, groups in which a second emotion is the emotion C, and groups in which a second emotion is the emotion D. In this case, an emotion class is a set of all the classes to which an emotion belongs. For example, a group to the emotion A after the synthesis is associated with an emotion class which is a set of all the classes to which the emotion A belongs.

The synthesis unit 12 may synthesize, into one group for each of the axes, groups of biological information pattern variation amounts associated with the emotion information in which the second emotion belongs to the same class on an axis. In this case, the synthesis unit 12 may synthesize, into one group, for example, the groups of biological information pattern variation amounts associated with emotion information including second emotions belonging to al. The emotions belonging to al are the emotion A and the emotion B. In this example, the synthesis unit 12 may synthesize, into one group, the groups of the biological information pattern variation amounts associated with the emotion information in which the second emotion is the emotion A or the emotion B. Similarly, the synthesis unit 12 may synthesize, into one group, the groups of biological information pattern variation amounts associated with emotion information including second emotions belonging to α2. Further, the synthesis unit 12 synthesizes, into one group, the groups of biological information pattern variation amounts associated with emotion information including second emotions to belong to β1. The emotions belonging to β1 are the emotion A and the emotion C. In this example, the synthesis unit 12 may synthesize, into one group, the groups of the biological information pattern variation amounts associated with the emotion information in which the second emotion is the emotion A or the emotion C. Similarly, the synthesis unit 12 may synthesize, into one group, the groups of biological information pattern variation amounts associated with emotion information including second emotions belonging to β2. In this case, the emotion class is a class on any of the axes. The groups after the synthesis are associated with any of the emotion classes on any of the axes. For example, a group of the biological information pattern variation amounts each associated with pieces of the emotion information including the second emotions belonging to al is associated with the emotion class that is the class α1.

The second distribution formation unit 13 forms, as described above, a probability density distribution for each of the groups after the synthesis by the synthesis unit 12. In the probability density distribution generated by the second distribution formation unit 13, ωᵢ in p(x|ωᵢ) described in the description of the comparative example is an element common to emotion information associated with biological information pattern variation amounts included in the same group after the synthesis. The common element is, for example, an emotion class. The common element that is an emotion class is, for example, a group of the predetermined number of classes to which emotions belong. The common element that is an emotion class may be, for example, the group of all classes to which emotions belong. The common element that is an emotion class may be, for example, the group of one class to which emotions belong. Like the description of the comparative example, x in this case is a biological information pattern variation amount. The second distribution formation unit 13 stores, in the learning result storage unit 14, a probability density distribution formed for each of the groups after the synthesis as the result of the learning.

Each component of the emotion recognition device 1 is also described as follows, for example, from the viewpoint of learning of a biological information pattern variation amount (hereinafter also referred to as “pattern”) associated with the emotion A by a supervised machine learning method. In the following description, emotions can also be classified into four emotions based on the two classes (α1 and α2) on the axis α, and on the two classes (β1 and β2) on the axis β. Similarly, the four emotions are the emotion A, the emotion B, the emotion C, and the emotion D. Similarly, the emotion A belongs to α1 and β1. The emotion B belongs to α 1 and β2. The emotion C belongs to α2 and β1. The emotion D belongs to α2 and β2.

The classification unit 10 records the information of biological information pattern variation amounts (i.e., relative changes) in the direction from α1 to α2 and the reverse direction thereof, and in the direction from β1 to β2 and the reverse direction thereof, based on the input emotion information from the input biological information patterns and emotion information. For example, when a change in emotion induced by a stimulus is a change from the emotion B to the emotion A, the obtained relative change in biological information pattern corresponds to a relative change from β2 to β1. For example, the relative change from β2 to β1 represents a biological information pattern variation amount (i.e., a relative value) obtained when a class on the axis β to which the emotion induced by the stimulus belongs is changed from β2 to β1. When the change in the emotion induced by the stimulus is a change from the emotion C to the emotion A, the obtained relative change in biological information pattern corresponds to a relative change from α2 to α1. When the change in the emotion induced by the stimulus is a change from the emotion D to the emotion A, the obtained relative change in biological information pattern corresponds to a relative change from α2 to α1 and from β2 to β1.

The first distribution formation unit 11 forms the classification results thereof in the forms of corresponding probability density distributions.

Further, the synthesis unit 12 extracts parts common to the changes in the biological information patterns described above. The synthesis unit 12 inputs the results thereof into the second distribution formation unit 13.

The second distribution formation unit 13 forms the probability density distribution of relative values common to changes to the emotion A (changes from α2 to α1 and changes from β2 to β1) based on the input common elements. The second distribution formation unit 13 stores the formed probability density distribution in the learning result storage unit 14.

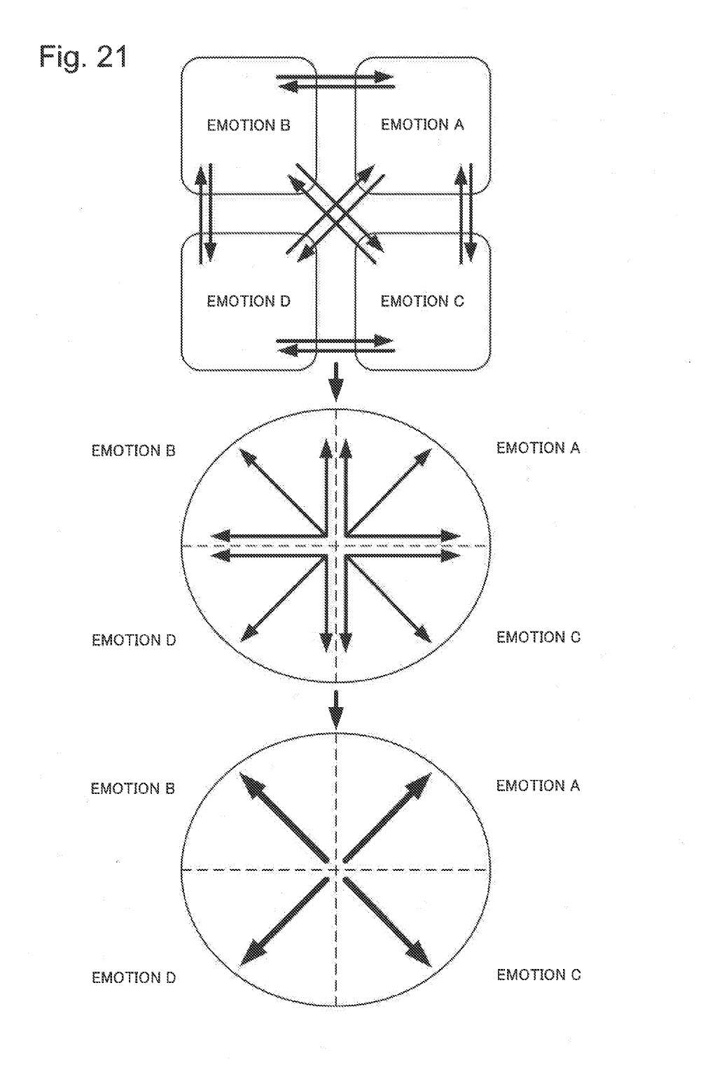

FIG. 21 is a view schematically representing processing of the first distribution formation unit 11, the synthesis unit 12, and the second distribution formation unit 13. The drawing illustrated in the upper section of FIG. 21 schematically represents biological information pattern variation amounts associated with various changes in emotion. The arrows in the drawing illustrated in the middle section of FIG. 21 schematically indicate the mean vectors of biological information pattern variation amounts included in groups each associated with changes in emotion. The arrows in the drawing illustrated in the lower section of FIG. 21 schematically represents vectors indicating mean values of biological information pattern variation amounts included in groups after synthesis. In the processing schematically illustrated in FIG. 21, note that, when the vectors of the relative changes to, for example, the emotion A are synthesized, the second distribution formation unit 13 does not simply synthesize all the changes to the emotion A but synthesizes the changes, for example, in order described below. A change in class to which an emotion belongs in a change from the emotion D to the emotion A is a change from α2 to α1 and from β2 to β1. A change in class to which an emotion belongs in a change from the emotion B to the emotion A is a change from β2 to β1. A change in class to which an emotion belongs in a change from the emotion C to the emotion A is a change from α2 to α1. Thus, on the assumption that the relative changes to the emotion A are relative changes from α2 to α1 and from β2 to β1, the second distribution formation unit 13 carries out synthesis, for example, in the order described below, to synthesize the vectors along the directions. The second distribution formation unit 13 first synthesizes the probability density distribution of changes from the emotion B to the emotion A and the probability density distribution of changes from the emotion C to the emotion A. The second distribution formation unit 13 synthesizes the result of synthesizing and the probability density distribution of changes from the emotion D to the emotion A.

The probability density distributions of changes to the emotion A synthesized in such a manner are expected to be outstanding (i.e., separated from the probability density distributions of the other emotions) in comparison with the comparative example in both of the direction of the α axis and the direction of the β axis.

The emotion recognition device 1 in an estimation phase is described next in detail with reference to the drawings.

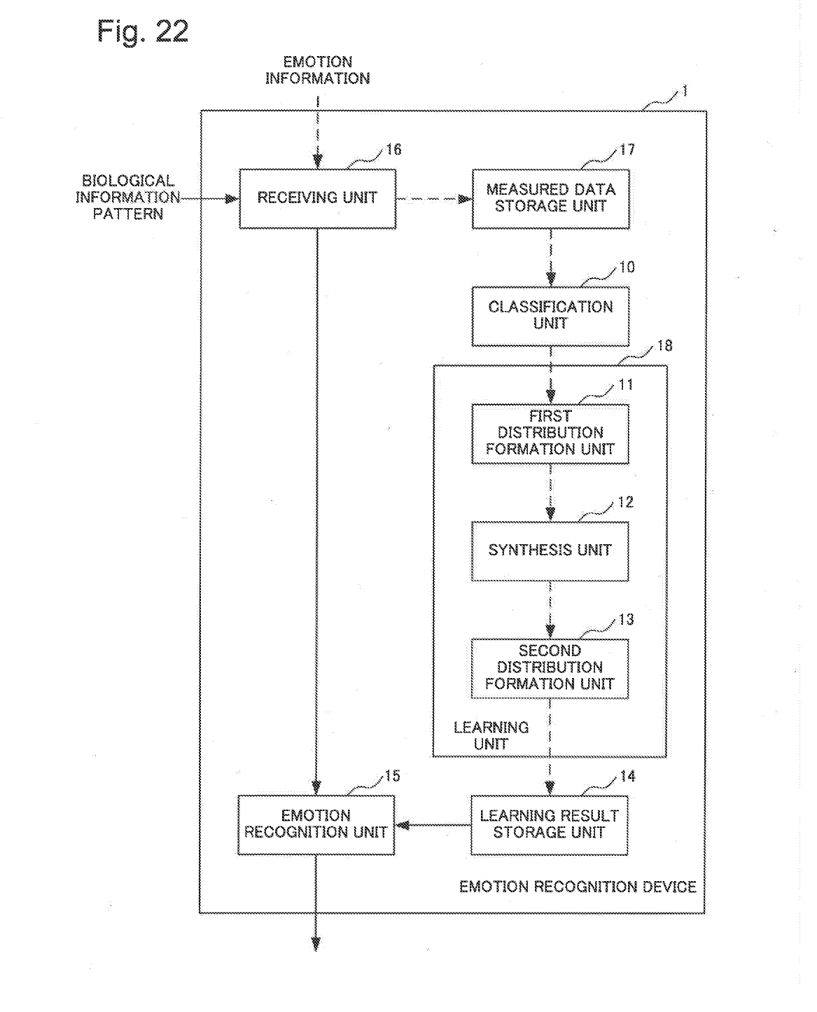

FIG. 22 is a block diagram representing an example of a configuration of the emotion recognition device 1 of the present exemplary embodiment in the estimation phase.

According to FIG. 22, a biological information pattern variation amount is input into the receiving unit 16 in the estimation phase. The receiving unit 16 receives the biological information pattern variation amount. However, the receiving unit 16 does not receive any emotion information in the estimation phase. In the estimation phase, the receiving unit 16 sends the received biological information pattern variation amount to the emotion recognition unit 15.

For example, an experimenter may provide instructions to switch from the learning phase to the estimation phase. Alternatively, the receiving unit 16 may determine that the phase is switched to the estimation phase when the biological information pattern variation amount is received and no emotion information is received. When the phase of the emotion recognition device 1 is switched to the estimation phase, the emotion recognition device 1 may switch the phase to the estimation phase after the learning unless the result of the learning is stored in the learning result storage unit 14.

The emotion recognition unit 15 receives the biological information pattern variation amount from the receiving unit 16. Using the learning result stored in the learning result storage unit 14, the emotion recognition unit 15 estimates an emotion induced by a stimulus applied to a test subject when the received biological information pattern variation amount is obtained. The emotion recognition unit 15 outputs the result of the estimation, i.e., the estimated emotion to, for example, the output unit 23. The result of the learning is, for example, the above-described probability distribution p(x|ωᵢ). In this case, the emotion recognition unit 15 derives the probability P(ωᵢ|x) for each of a plurality of emotion classes based on the expression shown in Math. 1. When the emotion class is the group of all the classes to which an emotion belongs, the emotion is specified by the emotion class ωᵢ. Hereinafter, the emotion specified by the emotion class ωᵢ is referred to as “the emotion ωᵢ”. The probability P(ωᵢ|x) represents a probability that the emotion ωᵢ is an emotion induced by a stimulus applied to a test subject when the received biological information pattern variation amount x is obtained. The emotion recognition unit 15 estimates that the emotion ωᵢ of which the derived probability P(ωᵢ|x) is the highest is an emotion induced by a stimulus applied to a test subject when the biological information pattern variation amount received by the emotion recognition unit 15 is obtained. The emotion recognition unit 15 may select, as the result of the emotion recognition, the emotion ωᵢ of which the derived probability P(ωᵢ|x) is the highest. When the emotion class ωᵢ is any one of the classes on any one of the axes, for example, the emotion recognition unit 15 may derive P(ωᵢ|x) for each of the emotion classes ωᵢ. On each of the axes, the emotion recognition unit 15 may select, from two classes on the axis, a class for which the higher P(ωᵢ|x) is derived. The emotion recognition unit 15 may select an emotion belonging to all the selected classes as the result of the emotion recognition.

The selected emotion is the emotion estimated as the emotion induced by the stimulus applied to the test subject when the biological information pattern variation amount received by the emotion recognition unit 15 is obtained. The emotion recognition unit 15 may output the estimated emotion as the result of the estimation. The emotion recognition unit 15 may send, for example, an emotion identifier of the estimated emotion to the output unit 23.

The operation of the emotion recognition system 2 of the present exemplary embodiment in a learning phase is described next in detail with reference to the drawings.

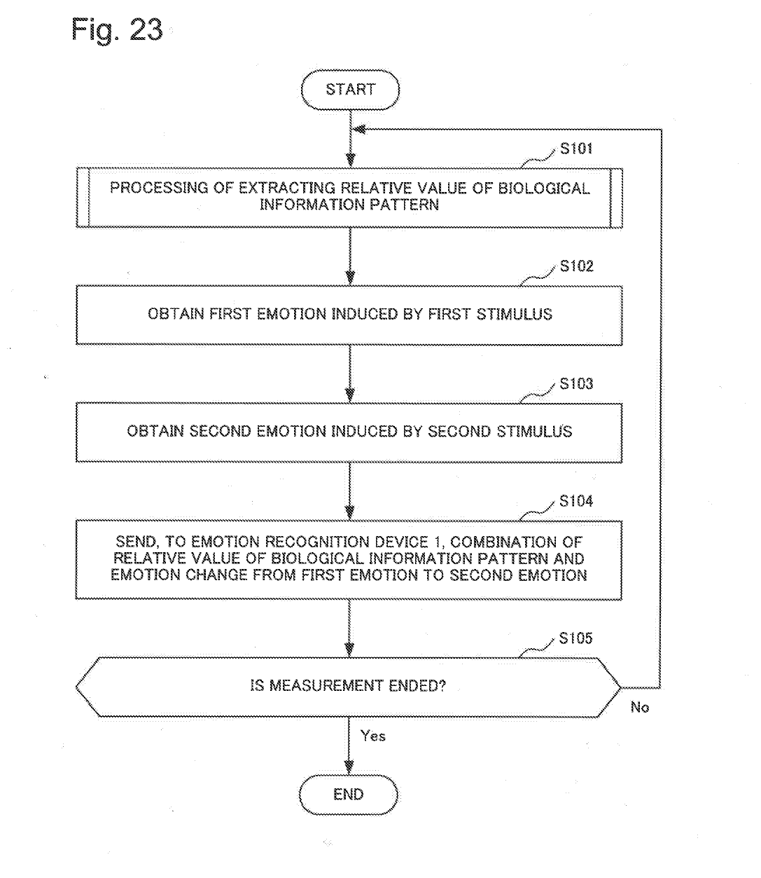

FIG. 23 is a flowchart representing an example of an operation of the emotion recognition system 2 of the present exemplary embodiment in the learning phase. The emotion recognition system 2 first carries out processing of extracting the relative value of a biological information pattern (step S101). The relative value of the biological information pattern is extracted by the processing of extracting the relative value of the biological information pattern. The processing of extracting the relative value of the biological information pattern is described in detail later. An experimenter inputs a first emotion induced by a first stimulus to the emotion recognition system 2 by the emotion input unit 22. The emotion input unit 22 acquires the first emotion induced by the first stimulus (step S102). The experimenter further inputs a second emotion induced by a second stimulus to the emotion recognition system 2 by the emotion input unit 22. The emotion input unit 22 acquires the second emotion induced by the second stimulus (step S103). The biological information processing unit 21 sends the relative value of the biological information pattern to the emotion recognition device 1. Further, the emotion input unit 22 sends, to the emotion recognition device 1, emotion information representing an emotion change from the first emotion to the second emotion. In other words, the emotion recognition system 2 sends, to the emotion recognition device 1, the combination of the relative value of the biological information pattern and the emotion change from the first emotion to the second emotion. When the measurement is ended (Yes in step S105), the emotion recognition system 2 ends the operation shown in FIG. 23. When the measurement is not ended (No in step S105), the operation of the emotion recognition system 2 returns to step S101.

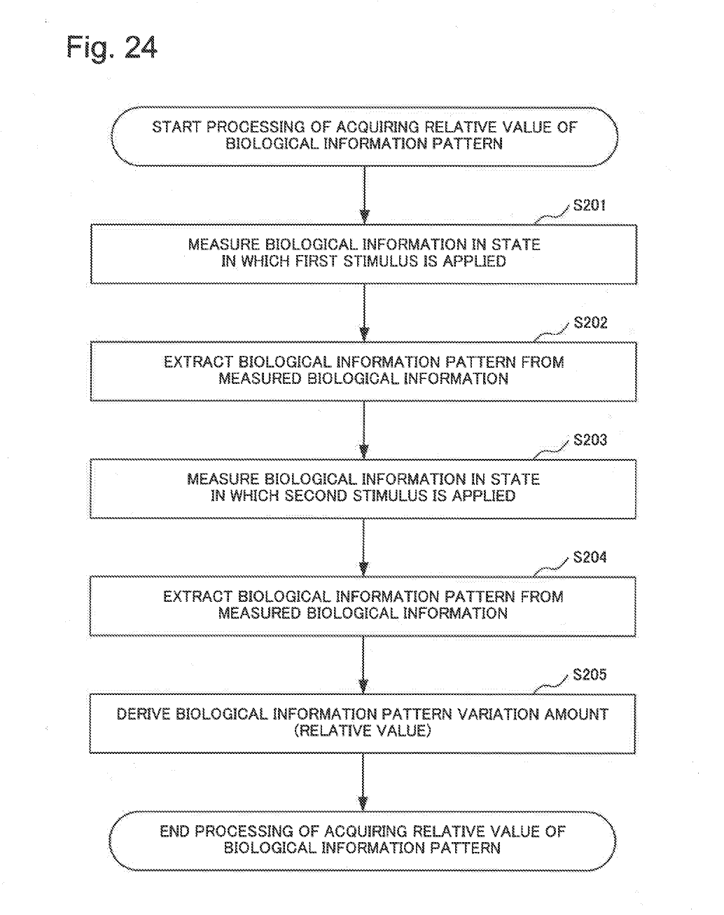

FIG. 24 is a flowchart representing an example of an operation of processing of extracting the relative value of a biological information pattern by the emotion recognition system 2 of the present exemplary embodiment. The sensing unit 20 measures biological information in the state where the first stimulus is applied (step S201). The biological information processing unit 21 extracts a biological information pattern from the biological information measured in step S201 (step S202). The sensing unit 20 further measures biological information in the state where the second stimulus is applied (step S203). The biological information processing unit 21 extracts a biological information pattern from the biological information measured in step S203 (step S204). The biological information processing unit 21 derives a biological information pattern variation amount (i.e. a relative value) from the biological information measured in step S202 and step S204 (step S205). The emotion recognition system 2 ends the operation shown in FIG. 24.

The sensing unit 20 may start the measurement of the biological information from the state where the first stimulus is applied, and may end the measurement of the biological information in the state where the second stimulus is applied. Meanwhile, the sensing unit 20 may continuously measure the biological information. The biological information processing unit 21 may specify, in the biological information measured by the sensing unit 20, the biological information measured in the state where the first stimulus is applied, and the biological information measured in the state where the second stimulus is applied. The biological information processing unit 21 can specify, by using various methods, the biological information measured in the state where the first stimulus is applied, and the biological information measured in the state where the second stimulus is applied. For example, the biological information processing unit 21 may specify a portion included within the predetermined fluctuation range for predetermined time period or more in the measured biological information. The biological information processing unit 21 may estimate that the portion specified in the first half of the measurement is the biological information measured in the state where the first stimulus is applied. The biological information processing unit 21 may estimate that the portion specified in the latter half of the measurement is the biological information measured in the state where the second stimulus is applied.

The operation of the emotion recognition device 1 of the present exemplary embodiment in a learning phase is described next in detail with reference to the drawings.

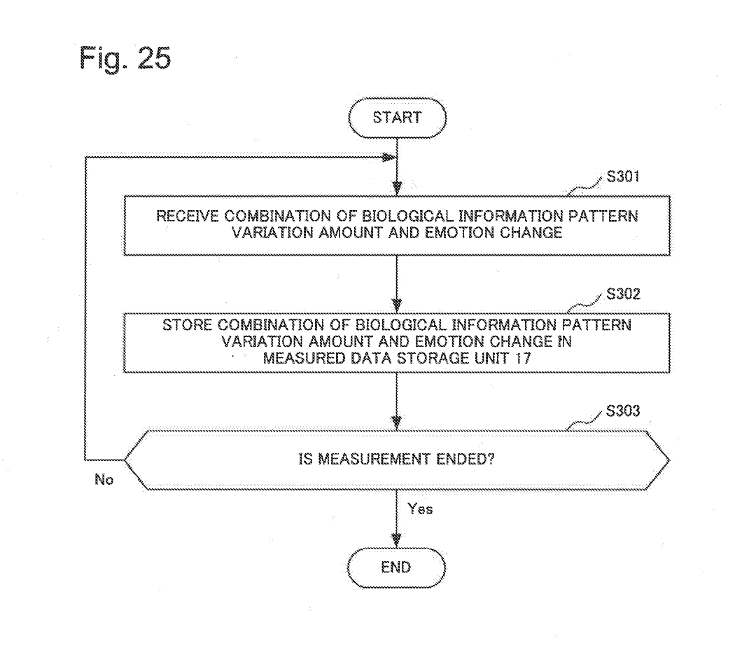

FIG. 25 is a first flowchart representing an example of an operation of the emotion recognition device 1 of the present exemplary embodiment in the learning phase. The receiving unit 16 receives the combination of a biological information pattern variation amount and an emotion change (step S301). The receiving unit 16 stores the combination of the biological information variation amount and the emotion change in the measured data storage unit 17 (step S302). When the measurement is ended (Yes in step S303), the emotion recognition device 1 ends the operation shown in FIG. 25. When the measurement is not ended (No in step S303), the operation of the emotion recognition device 1 returns to step S301.

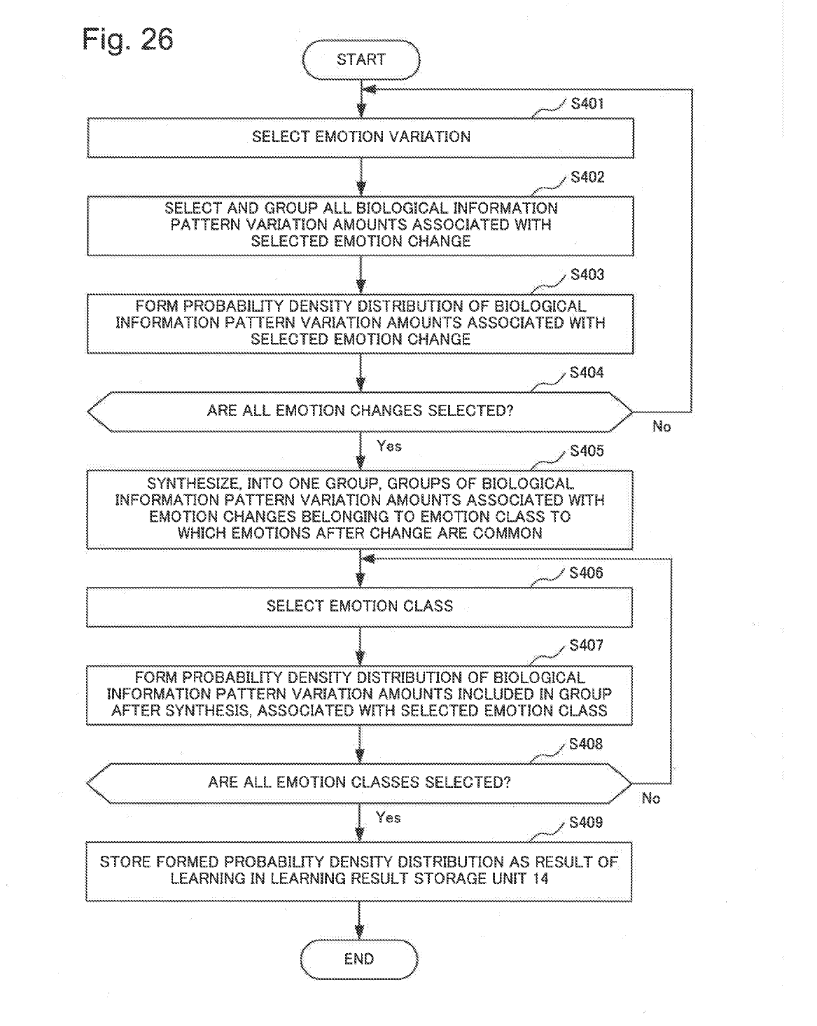

FIG. 26 is a first flowchart representing an example of the operation of the emotion recognition device 1 of the present exemplary embodiment in a learning phase. After the end of the operation shown in FIG. 25, the emotion recognition device 1 may start the operation shown in FIG. 26, for example, according to instructions by an experimenter.

The classification unit 10 selects one emotion change from emotion changes each associated with biological information pattern variation amounts stored in the measured data storage unit 17 (step S401). The classification unit 10 selects all the biological information pattern variation amounts associated with the selected emotion change (step S402). The classification unit 10 groups the biological information pattern variation amounts associated with the selected emotion change into one group. The first distribution formation unit 11 forms the probability density distribution of the biological information pattern variation amounts associated with the emotion change selected in step S401 based on the biological information pattern variation amounts selected in step S402 (step S403). When all the emotion changes are selected (Yes in step S404), the operation of the emotion recognition device 1 proceeds to step S405. When non-selected emotion variation is present (No in step S404), the operation of the emotion recognition device 1 returns to step S401.

In step S405, the synthesis unit 12 synthesizes, into one group, the groups of biological information pattern variation amounts associated with emotion changes of which emotions after a change belong to a common emotion class (step S405).

The second distribution formation unit 13 selects one emotion class (step S406). The second distribution formation unit 13 forms the probability density distribution of biological information pattern variation amounts included in groups after synthesis which are associated with the selected emotion class (step S407). When all emotion classes are selected (Yes in step S408), the operation of the emotion recognition device 1 proceeds to step S409. When an emotion class that is not selected exists (No in step S408), the operation of the emotion recognition device 1 returns to step S406.

In step S409, the second distribution formation unit 13 stores the formed probability density distribution as the result of the learning in the learning result storage unit 14. The emotion recognition device 1 ends the operation shown in FIG. 26. The second distribution formation unit 13 may carry out the operation of step S409 after the operation of step S407.

The operation of the emotion recognition system 2 of the present exemplary embodiment in an estimation phase is described next in detail with reference to the drawings.

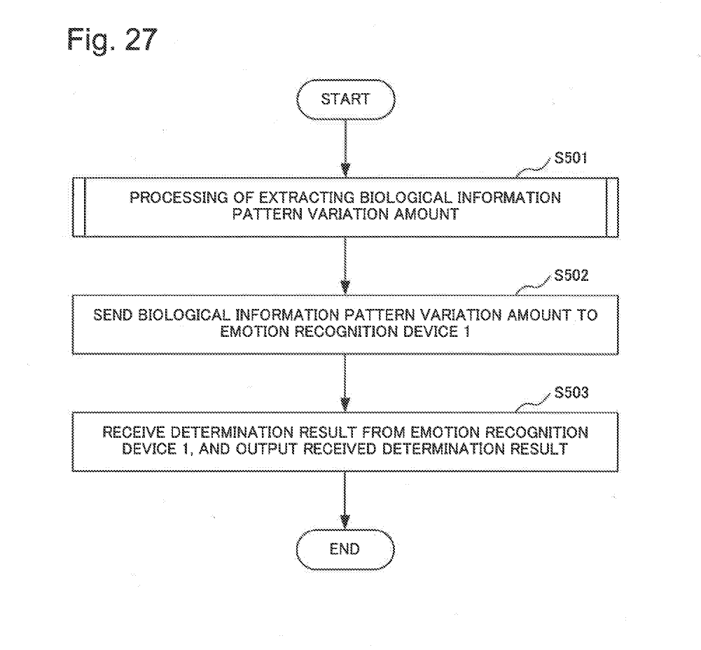

FIG. 27 is a flowchart representing an example of an operation of the emotion recognition system 2 of the present exemplary embodiment in the estimation phase. The emotion recognition system 2 first carries out processing of extracting a biological information pattern variation amount (step S501). The processing of extracting a biological information pattern variation amount in the estimation phase is the same as the processing of extracting a biological information pattern variation amount shown in FIG. 24. The biological information processing unit 21 sends the extracted biological information pattern variation amount to the emotion recognition device 1 (step S502). The emotion recognition device 1 estimates the second emotion of a test subject when the sent biological information pattern variation amount is obtained. The emotion recognition device 1 sends the determination result (i.e., an estimated second emotion) to the output unit 23. The output unit 23 receives the determination result from the emotion recognition device 1. The output unit 23 outputs the received determination result (step S503).

The operation of the emotion recognition device 1 of the present exemplary embodiment in an estimation phase is described next in detail with reference to the drawings.

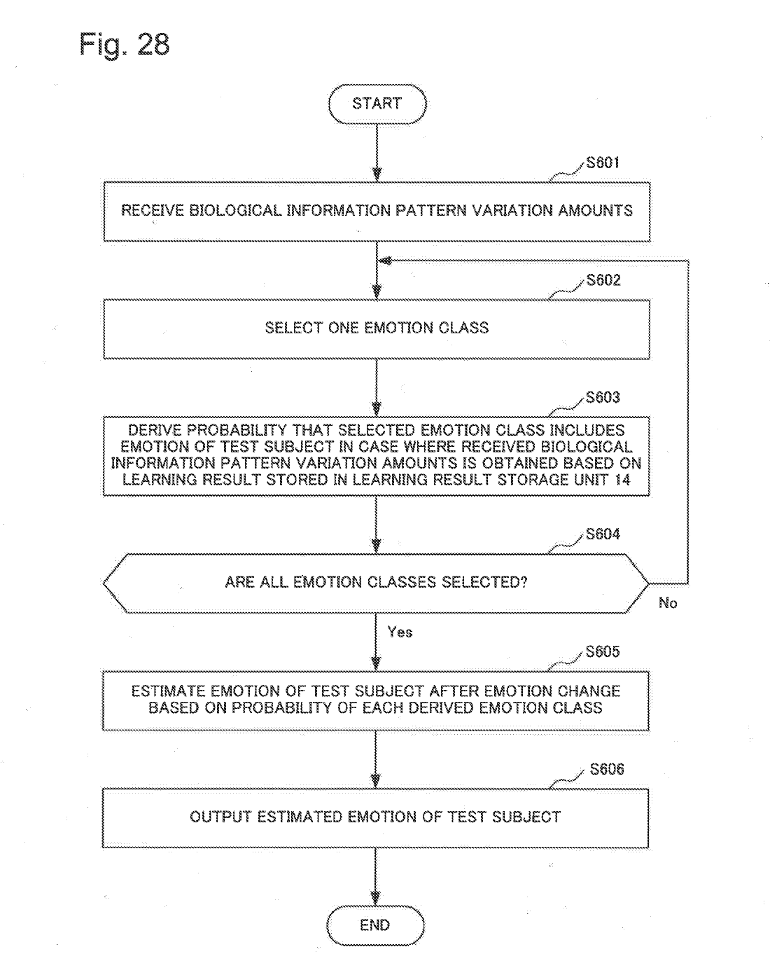

FIG. 28 is a flowchart representing an example of an operation of the emotion recognition device 1 of the present exemplary embodiment in the estimation phase. First, the receiving unit 16 receives a biological information pattern variation amount from the biological information processing unit 21 (step S601). In the estimation phase, the receiving unit 16 sends the received biological information pattern variation amount to the emotion recognition unit 15. The emotion recognition unit 15 selects one emotion class from a plurality of emotion classes (step S602). As described above, an emotion class is, for example, a group of one or more classes to which emotions belong. The emotion class may be an emotion. In this case, the plurality of emotion classes described above are a plurality of emotions determined in advance. In the present exemplary embodiment, an emotion is represented by a group of all the classes to which the emotion belongs. The emotion classes may be one of the classes on one of the axes by which emotions are classified. In this case, the plurality of emotion classes described above are all the classes on all the axes. The emotion recognition unit 15 derives a probability that the second emotion of a test subject when the received biological information pattern variation amount is obtained is included in the selected emotion class based on learning results stored in the learning result storage unit 14 (step S603). When an emotion class that is not selected exists (No in step S604), the emotion recognition device 1 repeats the operations from the operation of step S602. When all the emotion classes are selected (Yes in step S604), the emotion recognition device 1 carries out the operation of step S605.

In step S605, the emotion recognition unit 15 estimates the emotion of the test subject after variation in emotion (i.e., the second emotion) based on the derived probability of each of the emotion classes. When the emotion classes are emotions, the emotion recognition unit 15 may select, as the estimated emotion, an emotion of which the probability is the highest. When an emotion classes is one of the classes on one of the axes, for each axis, the emotion recognition unit 15 may select, for each of the axes, a class of which the probability is the highest from classes on an axis. In the present exemplary embodiment, as described above, an emotion is specified by classes selected on all the axes. The emotion recognition unit 15 may select, as the estimated emotion, an emotion specified by the selected emotion classes. The emotion recognition unit 15 outputs the estimated emotion of the test subject to the output unit 23.

The present exemplary embodiment described above has an advantage in that a decrease in the accuracy of identification of an emotion due to a fluctuation in emotion recognition reference can be suppressed. The reason thereof is because the learning unit 18 carries out learning based on a biological information pattern variation amount representing a difference between biological information obtained in the state where a stimulus for inducing a first emotion is applied and biological information obtained in the state where a stimulus for inducing a second emotion is applied. In other words, the learning unit 18 does not use biological information at rest as the reference of the biological information pattern variation amount. The biological information at rest fluctuates depending on an individual test subject and on the state of the test subject. Accordingly, the learning unit 18 may be prevented from the possibility of carrying out incorrect learning. Deterioration of the accuracy of identification of the emotion of the test subject, estimated based on the result of learning by the learning unit 18, can be suppressed by decreasing the possibility of the incorrect learning. In the following description, the effect of the present exemplary embodiment is described in more detail.

As described above, the state of a test subject directed to be at rest is not always equal. It is difficult to suppress the fluctuation of the emotion of the test subject directed to be at rest. Accordingly, a biological information pattern in a resting state is not always equal. Commonly, a biological information pattern in a resting state is considered to be the baseline of a biological information pattern, i.e., an emotion recognition reference. Therefore, it is difficult to suppress a fluctuation in emotion reference. In this case, there is a risk of learning of an incorrect pattern when a biological information pattern obtained from a test subject who should be in a resting state at learning is, for example, is similar to a biological information pattern in a state in which any emotion is induced. To cope with such a risk, a number of biological information patterns are given as learning data in the expectation that the biological information patterns at rest commonly approaches the state of averagely having no specific emotion by learning of a number of patterns.

The emotion recognition device 1 of the present exemplary embodiment carries out learning by using biological information pattern variation amounts obtained by separately applying stimuli for inducing two different emotions. The emotion recognition device 1 of the present exemplary embodiment does not use, in learning, data associated with biological information patterns obtained in a state in which a test subject is directed to be at rest. Accordingly, the emotion recognition device 1 of the present exemplary embodiment is not affected by a fluctuation in the state of the test subject directed to be at rest. Biological information patterns obtained in the state where a stimulus for inducing an emotion is applied is more stable than biological information patterns obtained in the state in which a test subject is directed to be at rest because an emotion is forcedly induced by the stimulus for inducing an emotion.

Accordingly, the emotion recognition device 1 of the present exemplary embodiment can reduce the risk of learning an incorrect pattern. Baseline biases caused by having specific emotions can be incorporated in advance by comprehensively learning variations from all emotions other than an emotion to be measured. It is not necessary to have expectation to eliminate the biases and to approach the state of having no specific emotion by a number of times of learning. Thus, learning by the emotion recognition device 1 of the present exemplary embodiment is efficient learning. In a case in which variation amounts from all emotions to emotions are learned in such a manner, even if a starting point is any emotion, an emotion being obtained by a test subject can be found when a biological information pattern variation amount in an estimation phase is obtained.

The superiority of a method of configuring a feature space in a learning phase and an estimation phase of the present exemplary embodiment is described in more detail below.

Commonly, a within-class variance and a between-class variance can be represented by equations described below. σ_{W}² shown in Math. 10 represents a within-class variance. σ_{B}² shown in Math. 11 represents a between-class variance.

$$\begin{matrix}{σ}_{W}^{2} = \frac{1}{n} {\sum }_{i = 1}^{c} {\sum }_{x ∈ {χ}_{i}} {( x - {m}_{i} )}^{t} ( x - {m}_{i} ) & [ Math . 10 ] \\ {σ}_{B}^{2} = \frac{1}{n} {\sum }_{i = 1}^{c} {n}_{i} {( {m}_{i} - m )}^{t} ( {m}_{i} - m ) & [ Math . 11 ]\end{matrix}$$

In the equations shown in Math. 10 and Math. 11, c represents the number of classes, n represents the number of feature vectors, m represents the mean of the feature vectors, mᵢ represents the mean of feature vectors belonging to a class i, and χᵢ represents the set of the feature vectors belonging to the class i. In the following description, a feature vector is also referred to as “pattern”.

It may be considered that in the feature space, the lower the within-class variance and the higher the between-class variance are, the higher the discriminability of the set of feature vectors is.

Accordingly, the magnitude of discriminability in the set of the feature vectors can be evaluated by a ratio J_{σ} defined by an equation shown in Math. 12.

$${J}_{σ} = \frac{{σ}_{B}^{2}}{{σ}_{W}^{2}} \qquad [ Math . 12 ]$$

It can be determined that the higher the ratio J_{σ} is, the superior the discriminability of the set of feature vectors is.

Discriminability in a case in which the number c of classes is two (c=2) will be described below.

A one-dimensional subspace which is only one straight line is defined as a one-dimensional subspace to which vectors m₁ and m₂ which are the mean vectors of the feature vectors belonging to the two classes commonly belong. The discriminability in the one-dimensional subspace is described below. Each pattern in the one-dimensional subspace is a projection, into which a feature vector is converted, on the one-dimensional subspace. For the sake of simplification, the coordinate of each of the patterns is displaced such that the centroid (i.e., the mean value) of all the patterns is represented by the zero point of the one-dimensional subspace. Accordingly, the mean value m of all the patterns is zero (m=0). The mean value m of all the patterns is the projection of the centroid of all the feature vectors into the one-dimensional subspace.

In the following description, the numbers of the patterns belonging to the two classes are equal. In other words, the number n₂ of the patterns belonging to the class 2 is equal to the number n₁ of the patterns belonging to the class 1 (n₂=n₁). Further, patterns belonging to one class are associated with patterns belonging to the other class on a one-to-one basis. Two patterns associated with each other are referred to as “x₁ⱼ” and “x₂ⱼ”. The pattern x₁ⱼ belongs to the class 1. The pattern x₂ⱼ belongs to the class 2. The value j is a number given to the combination of two patterns. The value j is any integer from 1 to nᵢ. In equations described below, m₁ is the mean value of the patterns belonging to the class 1, and m₂ is the mean value of the patterns belonging to the class 2. Because the mean value of all the patterns is zero, and the numbers of the patterns belonging to the two classes are equal to each other, the sum of m₁ and m₂ is zero.

In this case, σ_{W}², σ_{B}², and J_{σ} are represented by the equations shown in Math. 13, Math. 14, and Math. 15, respectively.

$$\begin{matrix}{σ}_{W}^{2} = \frac{1}{2 {n}_{1}} \{ {\sum }_{j = 1}^{{n}_{1}} {( {x}_{1 j} - {m}_{1} )}^{2} + {\sum }_{j = 1}^{{n}_{1}} {( {x}_{2 j} - {m}_{1} )}^{2} \} & [ Math . 13 ] \\ {σ}_{B}^{2} = \frac{1}{2 {n}_{1}} {n}_{1} ( {m}_{1}^{2} + {m}_{2}^{2} ) & [ Math . 14 ] \\ {J}_{σ} = \frac{{σ}_{B}^{2}}{{σ}_{W}^{2}} = \frac{{n}_{1} ( {m}_{1}^{2} + {m}_{2}^{2} )}{{\sum }_{j = 1}^{{n}_{1}} \{ {( {x}_{1 j} - {m}_{1} )}^{2} + {( {x}_{2 j} - {m}_{2} )}^{2} \}} & [ Math . 15 ]\end{matrix}$$

In the emotion recognition in the present exemplary embodiment, the patterns in the equations described above are defined as shown in Math. 16. In the present exemplary embodiment, the patterns x₁ⱼ and x₂ⱼ in the expressions described above can be substituted using Math. 16.

x₁ⱼ→x′₁ⱼ=x₁ⱼ−x₂ⱼ

x₂ⱼ→x′₂ⱼ=x₂ⱼ−x₁ⱼ [Math. 16]

Further, each pattern representing the centroid of each class is represented by Math. 17 based on the definitions. In the present exemplary embodiment, the patterns m₁ and m₂ representing the centroids in the expressions described above can be substituted using Math. 17.

m₁→m′₁=m₁−m₂

m₂→m′₂=m₂−m₁ [Math. 17]

Thus, a within-class variance σ_{W′}², a between-class variance σ_{B′}², and the ratio J_{σ}′ thereof in the present exemplary embodiment are represented by equations shown in the following Math. 18, Math. 19, and Math. 20, respectively.

$$\begin{matrix}\begin{matrix}{σ}_{w}^{′2} = \frac{1}{2 {n}_{1}} [ {\sum }_{j = 1}^{{n}_{1}} {\{ ( {x}_{1 j} - {x}_{2 j} ) - ( {m}_{1} - {m}_{2} ) \}}^{2} + \\ {\sum }_{j = 1}^{{n}_{1}} {\{ ( {x}_{2 j} - {x}_{1 j} ) - ( {m}_{2} - {m}_{1} ) \}}^{2} ] \\ = \frac{1}{2 {n}_{1}} [ 2 {\sum }_{j = 1}^{{n}_{1}} {\{ ( {x}_{1 j} - {x}_{2 j} ) - ( {m}_{1} - {m}_{2} ) \}}^{2} ]\end{matrix} & [ Math . 18 ] \\ \begin{matrix}{σ}_{B}^{′2} = \frac{1}{2 {n}_{1}} {n}_{1} \{ {( {m}_{1} - {m}_{2} )}^{2} + {( {m}_{2} - {m}_{1} )}^{2} \} \\ = \frac{1}{2 {n}_{1}} {n}_{1} \{ 2 {( {m}_{1} - {m}_{2} )}^{2} \}\end{matrix} & [ Math . 19 ] \\ {J}_{σ}^{′} = \frac{{σ}_{B}^{′2}}{{σ}_{W}^{′2}} = \frac{{n}_{1} \{ {( {m}_{1} - {m}_{2} )}^{2} \}}{{\sum }_{j = 1}^{{n}_{1}} {\{ ( {x}_{1 j} - {x}_{2 j} ) - ( {m}_{1} - {m}_{2} ) \}}^{2}} & [ Math . 20 ]\end{matrix}$$

A value obtained by multiplying the denominators of J_{σ} and J_{σ}′ by a value obtained by subtracting J_{σ} from J_{σ}′ is referred to as “ΔJ_{σ}”. ΔJ_{σ} is represented by the equation shown in Math. 20. Unless a state in which all the patterns of each class are equal to the mean value thereof is assumed, both of the denominators of J_{σ}′ and J_{σ} are greater than zero.

$$Δ {J}_{σ} = {\sum }_{j = 1}^{{n}_{1}} [ \{ {( {x}_{1 j} - {m}_{1} )}^{2} + {( {x}_{2 j} - {m}_{2} )}^{2} \} {( {m}_{1} - {m}_{2} )}^{2} - {\{ ( {x}_{1 j} - {x}_{2 j} ) - ( {m}_{1} - {m}_{2} ) \}}^{2} ( {m}_{1}^{2} + {m}_{2}^{2} ) ] \qquad [ Math . 21 ]$$

If the ΔJ is greater than zero, it can be concluded that the results of the emotion identification of the present exemplary embodiment are superior to those in common cases under the above-described premises such as the number of classes of c=2.

When the equation shown in Math. 20 is arranged using the relation shown in Math.21, ΔJ_{σ} is represented by an equation shown in Math. 22.

$$\begin{matrix}{s}_{1 j} = {x}_{1 j} - {m}_{1} \\ {s}_{2 j} = {x}_{2 j} - {m}_{2} & [ Math . 22 ] \\ Δ {J}_{σ} = {\sum }_{j = 1}^{{n}_{1}} ( {s}_{1 j}^{2} + {s}_{2 j}^{2} ) {( {m}_{1} - {m}_{2} )}^{2} - {( {s}_{1 j} - {s}_{2 j} )}^{2} ( {m}_{1}^{2} + {m}_{2}^{2} ) & [ Math . 23 ]\end{matrix}$$

ΔJ_{σj} in Math. 24 is the j-th element of ΔJ_{σ}, i.e., the j-th element of the right side of the equation shown in Math. 23. An equation shown in Math. 24 is derived by arranging a value obtained by dividing ΔJ_{σj} by s₂ⱼ² that is greater than zero.

$$\frac{Δ {J}_{σ j}}{{s}_{2 j}^{2}} = ( {m}_{2}^{2} - 2 {m}_{1} {m}_{2} ) {( \frac{{s}_{1 j}}{{s}_{2 j}} )}^{2} - 2 ( {m}_{1}^{2} + {m}_{2}^{2} ) ( \frac{{s}_{1 j}}{{s}_{2 j}} ) + ( {m}_{1}^{2} - 2 {m}_{1} {m}_{2} ) \qquad [ Math . 24 ]$$

The equation shown in Math. 24 is a quadratic equation with s₁ⱼ/s₂ⱼ. An equation m₁+m₂=0 holds because the mean value of all the patterns is zero. In other words, m₂ is equal to −m₁. Accordingly, m₂²−2m₁m₂ which is the coefficient of (s₁ⱼ/s₂ⱼ)² is greater than zero, as shown in Math. 25.

m₂²−2m₁m₂=3m₂²=3m₁²>0 [Math. 25]

Accordingly, ΔJ_{σj}/(s₂ⱼ²)>0, i.e., ΔJ_{σ}>0 is demonstrated if a relation shown in Math. 26 is established.

$$\frac{( {m}_{1}^{2} - 2 {m}_{1} {m}_{2} )}{( {m}_{2}^{2} - 2 {m}_{1} {m}_{2} )} - \frac{1}{4} {( \frac{2 ( {m}_{1}^{2} + {m}_{2}^{2} )}{( {m}_{2}^{2} - 2 {m}_{1} {m}_{2} )} )}^{2} > 0 \qquad [ Math . 26 ]$$

An expression shown in Math. 27 is derived by arranging the expression shown in Math. 26 reusing m₂=−m₁ which is a relation of m₂=−m₁ holding because the mean value of all the patterns is zero.

$$\frac{( {m}_{1}^{2} - 2 {m}_{1} {m}_{2} )}{( {m}_{2}^{2} - 2 {m}_{1} {m}_{2} )} - \frac{1}{4} {( \frac{2 ( {m}_{1}^{2} + {m}_{2}^{2} )}{( {m}_{2}^{2} - 2 {m}_{1} {m}_{2} )} )}^{2} = 1 - \frac{1}{4} {( \frac{4}{3} )}^{2} = \frac{5}{9} > 0 \qquad [ Math . 27 ]$$

In other words, it is shown that the emotion identification method of the present exemplary embodiment is superior in identification of classes (emotions) to the method in the comparative example under the premises such as the number of classes of c=2.

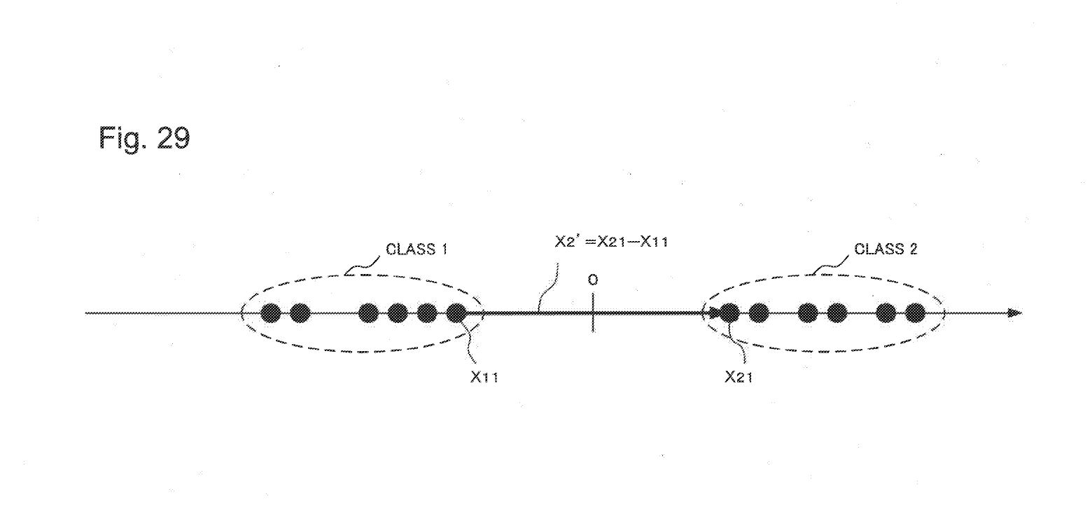

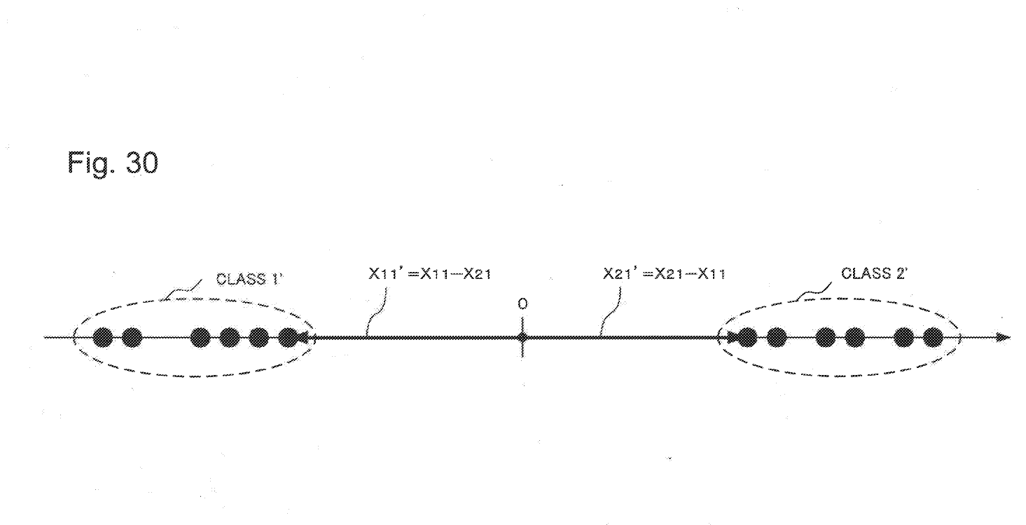

FIG. 29 and FIG. 30 are views schematically representing patterns in the comparative example and the present exemplary embodiment.

FIG. 29 is a view representing schematically patterns in a one-dimensional subspace in a feature space in the comparative example. The black circles illustrated in FIG. 29 are patterns in the comparative example. Patterns by the definition of the present exemplary embodiment are further illustrated in FIG. 29. A pattern in the present exemplary embodiment is defined as a difference between two patterns which is in the comparative example and is included in different classes.

FIG. 30 is a view schematically representing patterns in a one-dimensional subspace in a feature space in the present exemplary embodiment. The black circles illustrated in FIG. 30 represent patterns in a one-dimensional subspace in a feature space in the present exemplary embodiment, which are schematically drawn on the basis of the above-described definitions. As shown in FIG. 30, a distance in each distribution between classes, i.e., a between-class variance is relatively increased in comparison with a within-class variance. As a result, discriminability is improved. In the present exemplary embodiment, higher discriminability can be expected to be obtained with less learning data due to such superiority of discriminability. In the present exemplary embodiment, it can be directly estimated that the emotions of a test subject are any of emotions of which the number is more than two (for example, the emotions A, B, C, and D described above). However, the superiority of the present exemplary embodiment is conspicuous as described above, particularly in a case in which a class to which the emotions of the test subject belong is identified in two classes. Therefore, it may be considered to be preferable to adopt a method of determining 2ⁿ emotions in advance and identifying which of the 2ⁿ emotions the emotion of a test subject corresponds by repeating two-class identification n times as described above. The two-class identification represents identifying which of two classes the emotion of a test subject corresponds in a case where all the emotions are classified into the two classes. For example, when identifying which of the emotions A, B, C, and D the emotion of a test subject is by repeating the two-class identification twice, it is possible to identify which of the four emotions the emotion of the test subject is. In other words, the emotion of the test subject can be estimated.

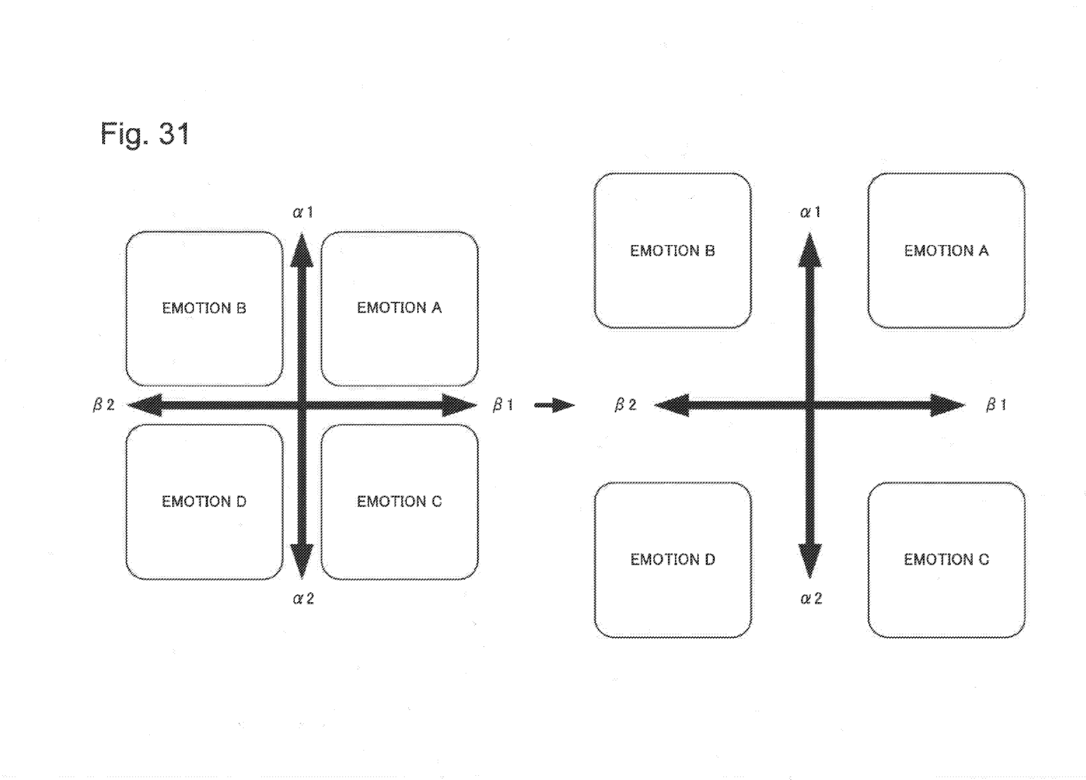

FIG. 31 is a view schematically representing distributions of biological information pattern variation amounts obtained for a plurality of emotions in the present exemplary embodiment. In an example shown in FIG. 31, all emotions are classified under four emotions by repeating two-class classification twice. As schematically illustrated in FIG. 31, in comparison with the comparative example, an effect that the distribution regions of the patterns is separated for the emotions A, B, C, and D is obtained by adopting classification in which two-class classification is repeated in the present exemplary embodiment. As a result, between-class classification becomes obviously easy in the present exemplary embodiment.

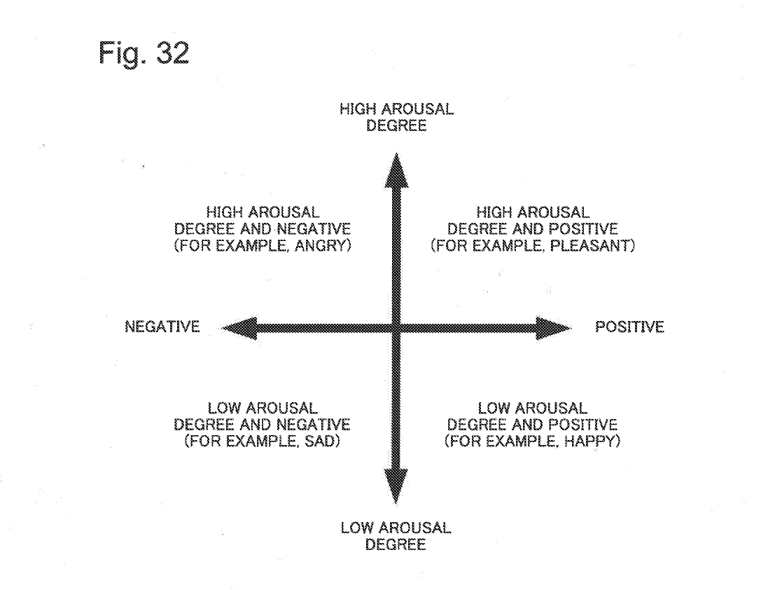

FIG. 32 is a view representing an example of classification of emotions. In the present exemplary embodiment, for example, a arousal degree and a valence evaluation (negative, positive) shown in FIG. 32 can be adopted as axes. Examples of classification of emotions shown in FIG. 32 are disclosed, for example, in NPL 2.

### Second Exemplary Embodiment

A second exemplary embodiment of the present invention is described next in detail with reference to the drawings.

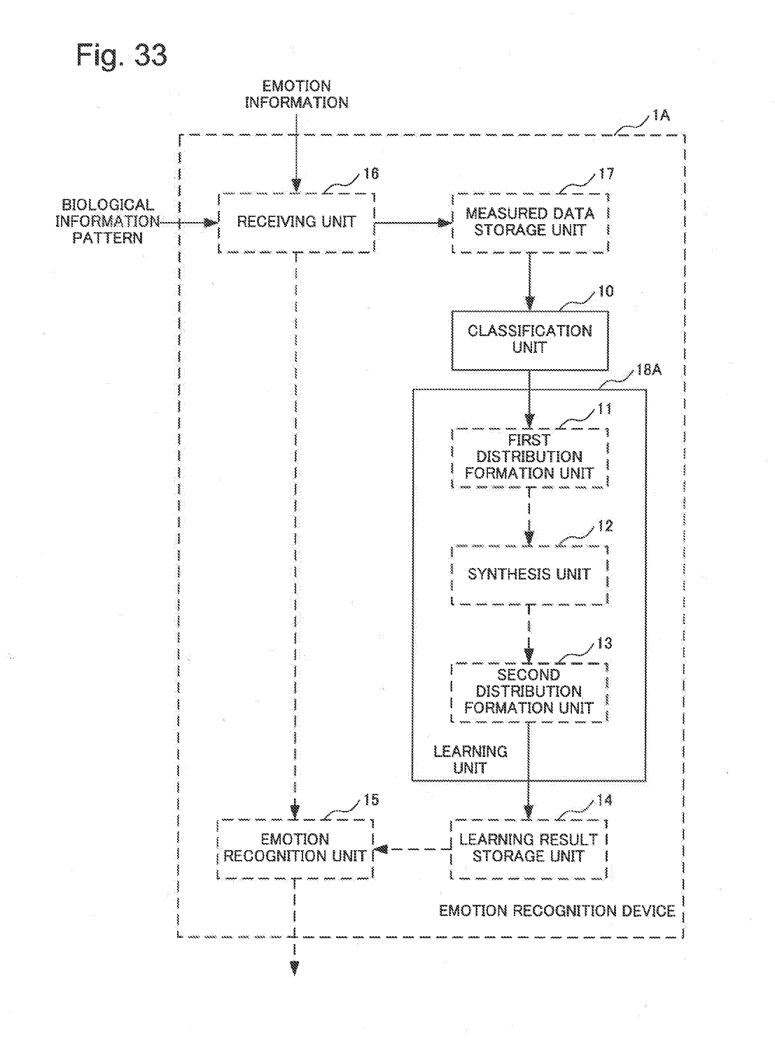

FIG. 33 is a block diagram representing a configuration of an emotion recognition device 1A of the present exemplary embodiment.

According to FIG. 33, the emotion recognition device 1A of the present exemplary embodiment includes a classification unit 10 and a learning unit 18A. The classification unit 10 classifies a biological information pattern variation amount representing a difference between first biological information and second biological information, obtained for a plurality of combinations of two different emotions (i.e., first emotion and second emotion) from among a plurality of emotions, based on the second emotion. The first biological information is biological information measured from a test subject by a means of sensing in the state in which a stimulus for inducing the first emotion is applied, which is one of the two emotions. The second biological information is biological information measured in the state in which a stimulus for inducing the second emotion is applied, which is the other of the two emotions, after the measurement of the first biological information. The learning unit 18A learns a relation between the biological information pattern variation amount and each of the plurality of emotions as the second emotion for which the biological information pattern variation amount is obtained, based on the result of classification of the biological information pattern variation amount. The learning unit 18A of the present exemplary embodiment may carry out, for example, learning in the same way as the learning unit 18 of the first exemplary embodiment of the present invention.

The present exemplary embodiment described above has the same effect as that of the first exemplary embodiment. The reason thereof is the same as the reason of generating the effect of the first exemplary embodiment.

### Other Exemplary Embodiment

Each of the emotion recognition device 1, the emotion recognition device 1A, and the emotion recognition system 2 can be implemented by using a computer and a program controlling the computer. Each of the emotion recognition device 1, the emotion recognition device 1A, and the emotion recognition system 2 can also be implemented by using dedicated hardware. Each of the emotion recognition device 1, the emotion recognition device 1A, and the emotion recognition system 2 can also be implemented by using a combination of a computer with a program controlling the computer, and dedicated hardware.

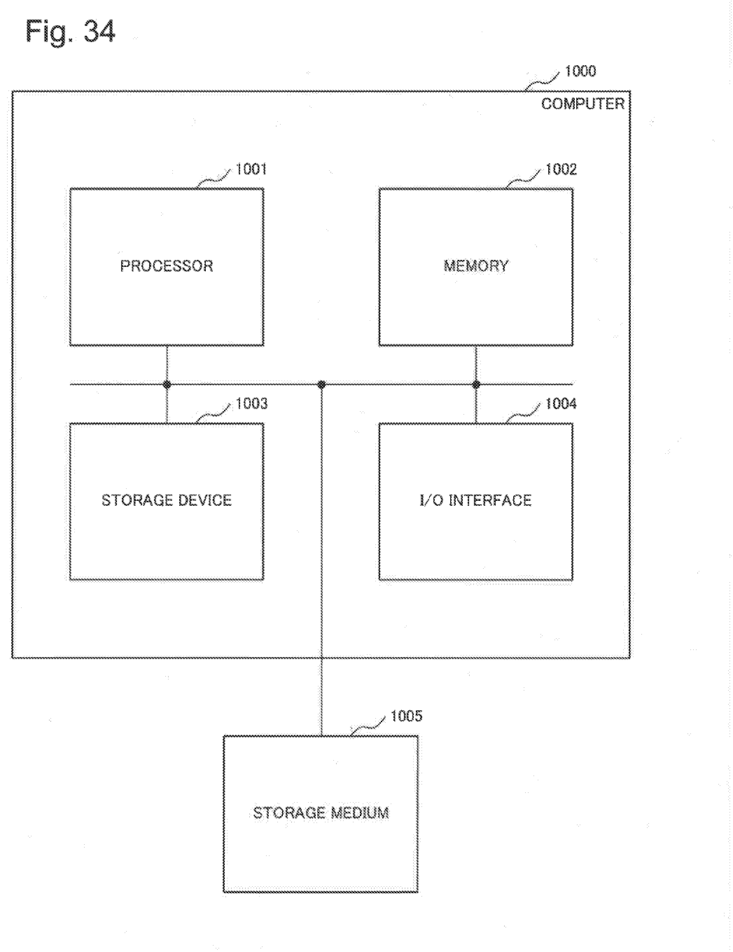

FIG. 34 is a view representing an example of a configuration of a computer 1000 with which the emotion recognition device 1, the emotion recognition device 1A, and the emotion recognition system 2 can be achieved. According to FIG. 34, the computer 1000 includes a processor 1001, a memory 1002, a storage device 1003, and an input/output (I/O) interface 1004. The computer 1000 can access a storage medium 1005. The memory 1002 and the storage device 1003 are, for example, storage devices such as a random access memory (RAM) and a hard disk. The storage medium 1005 is, for example, a storage device such as a RAM or a hard disk, a read only memory (ROM), or a portable storage medium. The storage device 1003 may be the storage medium 1005. The processor 1001 can read/write data and a program from/into the memory 1002 and the storage device 1003. The processor 1001 can access, for example, the emotion recognition system 2 or the emotion recognition device 1 through the I/O interface 1004. The processor 1001 can access the storage medium 1005. The storage medium 1005 stores a program that causes the computer 1000 to operate as the emotion recognition device 1, the emotion recognition device 1A, or the emotion recognition system 2.

The processor 1001 loads, into the memory 1002, the program that is stored in the storage medium 1005, and causes the computer 1000 to operate as the emotion recognition device 1, the emotion recognition device 1A, or the emotion recognition system 2. The processor 1001 executes the program loaded into the memory 1002, whereby the computer 1000 operates as the emotion recognition device 1, the emotion recognition device 1A, or the emotion recognition system 2.

Each of the units included in the following first group can be achieved by, for example, a dedicated program capable of achieving the function of each of the units, which is loaded into the memory 1002 from the storage medium 1005 that stores the program, and the processor 1001 that executes the program. The first group includes the classification unit 10, the first distribution formation unit 11, the synthesis unit 12, the second distribution formation unit 13, the emotion recognition unit 15, the receiving unit 16, the learning unit 18, the learning unit 18A, the biological information processing unit 21, the emotion input unit 22, and the output unit 23. Each of the units included in the following second group can be achieved by the memory 1002 included in the computer 1000 and/or the storage device 1003 such as a hard disk device. The second group includes the learning result storage unit 14 and the measured data storage unit 17. Alternatively, a part or all of the units included in the first group and the units included in the second group can also be achieved by a dedicated circuit that achieves the function of each of the units.

The present invention is described above with reference to the exemplary embodiments. However, the present invention is not limited to the exemplary embodiments described above. Various modifications that can be understood by those skilled in the art in the scope of the present invention can be made in the constitution and details of the present invention.

This application claims priority based on Japanese Patent Application No. 2014-109015, which was filed on May 27, 2014, and the entire disclosure of which is incorporated herein.

### REFERENCE SIGNS LIST

1 Emotion recognition device
1A Emotion recognition device
2 Emotion recognition system
10 Classification unit
11 First distribution formation unit
12 Synthesis unit
13 Second distribution formation unit
14 Learning result storage unit
15 Emotion recognition unit
16 Receiving unit
17 Measured data storage unit
18 Learning unit
20 Sensing unit
21 Biological information processing unit
22 Emotion input unit
23 Output unit
101 Emotion recognition device
110 Classification unit
114 Learning result storage unit
115 Emotion recognition unit
116 Receiving unit
117 Measured data storage unit
118 Learning unit
201 Emotion recognition system
220 Sensing unit
221 Biological information processing unit
222 Emotion input unit
223 Output unit
1000 Computer
1001 Processor
1002 Memory
1003 Storage device
1004 I/O interface
1005 Storage medium

1. An emotion recognition device comprising:
a memory that stores a set of instructions; and
at least one processor configured to execute the set of instructions to:
classify, based on a second emotion, a biological information pattern variation amount indicating a difference between first biological information and second biological information, the first biological information being measured by sensor from a test subject in a state in which a stimulus for inducing a first emotion is applied, the first emotion being one of two emotions obtained from a plurality of combinations of two different emotions from among a plurality of emotions, the second biological information being measured in a state in which a stimulus for inducing the second emotion which is the other of the two emotions is applied after the first biological information is measured; and
learn a relation between the biological information pattern variation amount and each of the plurality of emotions as the second emotion in a case where the biological information pattern variation amount is obtained, based on a result of classification of the biological information pattern variation amount.

2. The emotion recognition device according to claim 1, comprising:
the at least one processor is configured to:
estimate the second emotion in a case where the biological information pattern variation amount is measured based on the biological information pattern variation amount and the learned relation when the biological information pattern variation amount is given.

3. The emotion recognition device according to claim 2, wherein
the at least one processor is configured to:
classify the biological information pattern variation amount, for each of one or more class classifications, based on classes into which the second emotion in a case where the biological information pattern variation amount is measured is classified by the one or more class classifications each of which classifies an emotion in the plurality of emotions into one of two classes,
each of the plurality of emotions being specified by the classes into which the plurality of emotions are classified;
learn a relation between the biological information pattern variation amount and the classes into which the second emotion in a case where the biological information pattern variation amount is obtained is classified by the class classifications, for each of the one or more class classifications; and
estimate the second emotion by estimating the classes into which the biological information pattern variation amount is classified by the class classifications, for each of the one or more class classifications.

4. An emotion recognition system including the emotion recognition device according to claim 1, the emotion recognition system comprising:
the sensor;
a memory that stores a set of instructions; and
at least one processor configured to execute the set of instructions to:
derive the biological information pattern variation amount based on a difference between the first biological information measured in a state in which a stimulus for inducing the first emotion is applied and the second biological information measured in a state in which a stimulus for inducing the second emotion which is the other of the two emotions after the first biological information is applied is measured;
receive the first emotion and the second emotion; and
output an estimated emotion.

5. An emotion recognition method comprising:
classifying, based on a second emotion, a biological information pattern variation amount indicating a difference between first biological information and second biological information, the first biological information being measured by sensor from a test subject in a state in which a stimulus for inducing a first emotion is applied, the first emotion being one of two emotions obtained from a plurality of combinations of two different emotions from among a plurality of emotions, the second biological information being measured in a state in which a stimulus for inducing the second emotion which is the other of the two emotions is applied after the first biological information is measured; and
learning a relation between the biological information pattern variation amount and each of the plurality of emotions as the second emotion in a case where the biological information pattern variation amount is obtained, based on a result of classification of the biological information pattern variation amount.

6. The emotion recognition method according to claim 5, comprising:
estimating the second emotion in a case where the biological information pattern variation amount is measured based on the biological information pattern variation amount and the learned relation when the biological information pattern variation amount is given.

7. The emotion recognition method according to claim 6, wherein
the classifying includes classifying the biological information pattern variation amount is classified, for each of one or more class classifications, based on classes into which the second emotion in a case where the biological information pattern variation amount is measured is classified by the one or more class classifications each of which classifies the plurality of emotions into one of two classes;
each of the plurality of emotions is specified by the classes into which the plurality of emotions are classified;
the learning includes learning a relation between the biological information pattern variation amount and the classes into which the second emotion in a case where the biological information pattern variation amount is obtained is classified by the class classifications, for each of the one or more class classifications; and
the estimation includes estimating the second emotion by estimating the classes into which the biological information pattern variation amount is classified by the class classifications, for each of the one or more class classifications.

8. A non-transitory computer-readable storage medium storing an emotion recognition program that causes a computer to execute:
classification processing of classifying, based on a second emotion, a biological information pattern variation amount indicating a difference between first biological information and second biological information, the first biological information being measured by sensor from a test subject in a state in which a stimulus for inducing a first emotion is applied, the first emotion being one of two emotions obtained from a plurality of combinations of two different emotions from among a plurality of emotions, the second biological information being measured in a state in which a stimulus for inducing the second emotion which is the other of the two emotions is applied after the first biological information is measured; and
learning processing of learning a relation between the biological information pattern variation amount and each of the plurality of emotions as the second emotion in a case where the biological information pattern variation amount is obtained, based on a result of classification of the biological information pattern variation amount.

9. The non-transitory computer-readable storage medium according to claim 8, the storage medium storing an emotion recognition program that causes a computer to operate as:
emotion recognition processing of estimating the second emotion in a case where the biological information pattern variation amount is measured based on the biological information pattern variation amount and the learned relation when the biological information pattern variation amount is given.

10. The non-transitory computer-readable storage medium according to claim 9, wherein
the classification processing classifies the biological information pattern variation amount for each of one or more class classifications based on the class, into which the second emotion in a case where the biological information pattern variation amount is measured is classified, by the one or more class classifications each of which classifies an emotion in the plurality of emotions into one of two classes, each of the plurality of emotions being specified by the classes into which the plurality of emotions are classified;
the learning processing learns a relation between the biological information pattern variation amount and the classes into which the second emotion in a case where the biological information pattern variation amount is obtained is classified by the class classifications, for each of the one or more class classifications; and
the emotion recognition processing estimates the second emotion by estimating the classes into which the biological information pattern variation amount is classified by the class classifications, for each of the one or more class classifications.
